(19) United States
(12) Patent Application Publication
(10) Pub. No.: US 2024/0164789 A1
(43) Pub. Date: May 23, 2024

(54) OCCLUDING STENT, IMPLANTER THEREOF, AND IMPLANTING METHOD THEREOF

(71) Applicants: MICRO-TECH (NANJING) CO., LTD., Nanjing (CN); JIANGSU PROVINCE HOSPITAL, Nanjing (CN)

(72) Inventors: Guoxin ZHANG, Nanjing (CN); Jiwang WANG, Nanjing (CN); Jianyu WEI, Nanjing (CN); Yun WANG, Nanjing (CN); Lurong LI, Nanjing (CN); Weifeng ZHANG, Nanjing (CN); Huaiming SANG, Nanjing (CN); Zhenghua SHEN, Nanjing (CN); Changqing LI, Nanjing (CN); Derong LENG, Nanjing (CN)

(21) Appl. No.: 18/427,671

(22) Filed: Jan. 30, 2024

Related U.S. Application Data
(63) Continuation-in-part of application No. 17/059,217, filed on Nov. 26, 2020.

(30) Foreign Application Priority Data
Mar. 5, 2019 (CN) 201910164284.0

(51) Int. Cl. A61B 17/12; A61B 17/00

(52) U.S. Cl. CPC A61B 17/12172; A61B 17/0057; A61B 2017/00623; A61B 2017/00632; A61B 2017/12095

(57) ABSTRACT

Disclosed is an occluding stent. The occluding stent includes a distal flange occluding body, a proximal flange occluding body, and a connection portion. External surfaces of the distal flange occluding body and the proximal flange occluding body are both provided with a coating. An external surface of the connection portion is wholly or partially provided with a coating. A first occluding coating is provided between the distal flange occluding body and an inner cavity of the connection portion. The present application overcomes the defect that the existing occluder device for closure of an esophagobronchial fistula is harmful to surrounding tissues, makes the device retractable, enhances the safety of the device, and improves life and treatment of patients. A new safe and effective treatment method for an esophagobronchial fistula is provided.

### CROSS-REFERENCE TO RELATED APPLICATIONS

This application is a continuation-in-part of U.S. application Ser. No. 17/059,217, which is a national stage of International Application No. PCT/CN2020/077659, filed on Mar. 3, 2020. The International Application claims priority to Chinese Patent Application No. 201910164284.0, filed on Mar. 5, 2019. All of the aforementioned applications are hereby incorporated by reference in their entireties.

### TECHNICAL FIELD

The present application relates to the field of minimally invasive technologies, and in particular, to an occluding stent, an implanter thereof, and an implanting method thereof.

### BACKGROUND

In 2006, the first case of an atrial septal occluder used for closure of a 5 mm esophagobronchial fistula was reported in Paulo RS and so on, and the patient left the hospital 5 days after the surgery. Closure from the trachea via a tube causes small invasion, the operation is easy, and the effect is significant. Later, numerous umbrella-shaped devices and other similar mechanical occluding devices are developed based on the atrial septal defect occluder to close esophagobronchial fistulas. However, such devices still have the following disadvantages: (1) The occluder has end covers at proximal and distal ends, or even if the occluder is concave inwards, once the patient coughs, it causes the occluder to be pear-shaped, which easily damages surrounding tissues and causes massive hemorrhage or the like, leading to sudden death. (2) The occluder is liable to corrosion; besides, as a foreign body, the occluder may cause inflammatory reaction, and common occluding devices cannot resist the inflammatory reaction. (3) There is no withdrawal unit; once the occluder is implanted, it cannot be taken out through conservative medical therapy. (4) Generally, the occluder is implanted through the trachea, which affects the rescue channel; in addition, the occluder cannot be implanted under the direct vision of a doctor at any time, which causes certain risks. (5) The treatment method of expanding the esophagus circumferentially by using a conventional stent severely affects the daily life of the patient, causes long-term pain, and affects the life quality of the patient. (6) The occluder itself is heavy; the occluder compresses surrounding tissues, which causes avascular necrosis, or the occluder may even fall off and block the trachea, causing death.

### SUMMARY

An object of the present application is to provide an occluding stent, an implanter thereof, and an implanting method thereof, to solve many defects in the background. The present application overcomes the defect that the existing occluding device for closure of an esophagobronchial fistula is harmful to surrounding tissues, makes the device retractable, enhances the safety of the device, and improves life and treatment of patients. A new safe and effective treatment method for an esophagobronchial fistula is provided.

To achieve the foregoing objectives, the present application provides the following technical solutions.

An occluding stent, comprising a distal flange occluding body, a proximal flange occluding body, and a connection portion.

The distal flange occluding body and the proximal flange occluding body are respectively connected to two ends of the connection portion in a longitudinal direction A. In the longitudinal direction A of the occluding stent, a distalmost end of the distal flange occluding body, i.e., an end surface of a first occluding portion end of the distal flange occluding body, is flat when viewed in a radial direction B (i.e., a transverse direction or a vertical direction) of the occluding stent and has no outward protrusion in the longitudinal direction A away from the connection portion. Similarly, a proximalmost end of the proximal flange occluding body, i.e., an end surface of a second occluding portion end of the proximal flange occluding body, is flat when viewed in a radial direction B (i.e., a transverse direction or a vertical direction) of the occluding stent and has no outward protrusion in the longitudinal direction A away from the connection portion. The radial direction B may be parallel to the end surface of the first occluding portion end of the distal flange occluding body and the end surface of the second occluding portion end of the proximal flange occluding body, i.e., the transverse direction of the occluding stent. The transverse direction and the longitudinal direction of the occluding stent are perpendicular to each other, in other words, the radial direction B may be the radial direction (i.e., the transverse direction) of the distal flange occluding body, the proximal flange occluding body, and the connecting portion, and may be perpendicular to the longitudinal direction of the distal flange occluding body, the proximal flange occluding body, and the connecting portion, and the radial direction B may be perpendicular to the longitudinal direction A. Accordingly, the proximal end and the distal end of the occluding stent do not have end covers or tails protruding outwardly, thereby preventing damage to surrounding tissues.

The distal flange occluding body and proximal flange occluding body are respectively connected to two ends of the connection portion in the longitudinal direction A. In the longitudinal direction A of the occluding stent, an end surface of a distalmost end of the distal flange occluding body and an end surface of a proximalmost end of the proximal flange occluding body are both configured to have an opening at a middle portion of each end surface, i.e., the middle portion of each end surface is opened, such that an edge of the opening is at a distance from a center axis C in the longitudinal direction A of the occluding stent.

In one embodiment, the occluding stent of the present application may be of axisymmetric construction, the central axis C is an axis of symmetry passing through the occluding stent in the longitudinal direction A of the occluding stent. Further, the distal flange occluding body, the proximal flange occluding body, and the connecting portion are also axisymmetric structures, and the above-described center axis C is also an axis of symmetry passing through the distal flange occluding body, the proximal flange occluding body, and the connecting portion in the longitudinal direction A of the occluding stent.

The openings on the end surface of the distalmost end of the distal flange occluding body and on the end surface of the proximalmost end of the proximal flange occluding body may have various shapes, such as circle, square, etc., and the openings are formed around the center axis C.

In one embodiment, the distances from the edges of the openings on the end surface of the distalmost end of the distal flange occluding body and on the end surface of the proximalmost end of the proximal flange occluding body to the center axis C in the longitudinal direction A of the occluding stent is greater than 0 and less than or equal to 23 mm, and in one embodiment the distance is greater than or equal to 1 mm and less than or equal to 20 mm, or greater than or equal to 2 mm and less than or equal to 17 mm. The size of the openings in the middle portions of the end surface of the distalmost end of the distal flange occluding body and the end surface of the proximalmost end of the proximal flange occluding body may be set in the above ranges.

The connection portion may be a tubular structure, and the distal flange occluding body and the proximal flange occluding body are respectively connected to two ends of the connection portion in the longitudinal direction A. In one embodiment, the connection of the distal flange occluding body and the proximal flange occluding body to the connection portion may be a one-piece connection. The distal flange occluding body, the proximal flange occluding body, and the connecting portion may be a braided structure of metal wires, and the distal flange occluding body, the proximal flange occluding body, and the connecting portion may be formed by continuous braiding. The end surface of the distalmost end of the distal flange occluding body and the end surface of the proximalmost end of the proximal flange occluding body are not closed up inwardly along the radial direction B, and thus do not converge to the center axis C to form continuous braided end surfaces. The end surfaces have the shape of an opening, i.e. the middle portions of the end surfaces are empty and are not braided, the empty portions are formed around the center axis C. The braided wires (the braided wires in the shape of a circular ring) forming the openings (e.g., circular ring shape) are located at a certain distance away from the center axis C of the occluding stent. Therefore, there is no protruding tails on the end surfaces of the distalmost and proximalmost ends of the distal and proximal flange occluding bodies, formed by ponytail-like tying up of metal wires braided to the middle portions of end surfaces. Spaces among the metal wires of the braided structure may be holes, i.e., meshes. The mesh sizes of the braided structures of the distal flange occluding body, the proximal flange occluding body, and the connecting portion are the same or different. In one embodiment, the meshes of the braided distal flange occluding body and the proximal flange occluding body are larger than the mesh of the mesh structure of the connection portion.

A portion of the distal flange occluding body extending beyond the connection portion in the radial direction B perpendicular to the longitudinal direction A is formed as a first shoulder, and a portion of the proximal flange occluding body extending beyond the connection portion in the radial direction B is formed as a second shoulder, such that the occluding stent is configured such that the connection portion is located in a fistula, and the first shoulder of the distal flange occluding body and the second shoulder of the proximal flange occluding body are seated on two side walls of the fistula respectively to seal the fistula.

The height h1 of the first shoulder in the radial direction B allows the distal flange occluding body to be securely seated on the side wall of the fistula. In one embodiment, the height h1 of the first shoulder in the radial direction B is in the range of 2-10 mm. In one embodiment, the height h1 of the first shoulder in the radial direction B is in the range of 2-5 mm. In one embodiment, the height h1 of the first shoulder in the radial direction B is 3 mm.

The height h2 of the second shoulder in the radial direction B allows the proximal flange occluding body to be securely seated on the sidewall of the fistula. In one embodiment, the height h2 of the second shoulder in the radial direction B is in the range of 2-10 mm. In one embodiment, the height h2 of the second shoulder in the radial direction B is in the range of 2-5 mm. In one embodiment, the height h2 of the second shoulder in the radial direction B is 3 mm.

The height h1 of the first shoulder in the radial direction B is the same as or different from the height h2 of the second shoulder in the radial direction B, or h1 is greater than h2, or h1 is less than h2.

The angle α1 formed by the distal flange occluding body extending beyond the connection portion along the radial direction B relative to the connection portion 62 along the longitudinal direction A and the angle α2 formed by the proximal flange occluding body extending beyond the connection portion along the radial direction B relative to the connection portion along the longitudinal direction A may be any angle, for example, greater than 90 degrees, equal to 90 degrees, or less than 90 degrees, as long as the distal flange occluding body and the proximal flange occluding body can be securely seated on two side walls of the fistula to seal the fistula.

In one embodiment, the distal flange occluding body is umbrella-shaped, mushroom cap-shaped, cup-shaped, disc-shaped, bowl-shaped, hemi-spherical or spherical, the proximal flange occluding body is umbrella-shaped, mushroom cap-shaped, cup-shaped or spherical. Therefore, the cross-sections formed by cutting the distal flange occluding body and the proximal flange occluding body in the radial direction B may be in the shape of a circular ring. The end surface of the distalmost end of the distal flange occluding body and the end surface of the proximalmost end of the proximal flange occluding body are both configured to have an opening at a middle portion of each end surface, for example, a circular opening. A sealed occlusion state is formed between the distal flange occluding body and the proximal flange occluding body by using a coating.

Where a surface of the connection portion is wholly or partially provided with a coating; a surface of the distal flange occluding body is wholly or partially provided with a coating; and a surface of the proximal flange occluding body is wholly or partially provided with a coating.

Where a first occluding coating is provided between the distal flange occluding body and an inner cavity of the connection portion, and/or a second occluding coating is provided between the proximal flange occluding body and the inner cavity of the connection portion. In one embodiment, the first occluding coating and the second occluding coating are disposed on end surfaces of the distalmost and proximalmost ends (in the longitudinal direction A of the occluding stent) of the internal cavity of the connection portion along a direction parallel to the radial direction of the occluding stent to seal the end surfaces. Therefore, food or foreign objects are prevented from passing through the occluding stent, and entering from one alimentary canal into another alimentary canal, from one airway into one alimentary canal, from one alimentary canal into one airway, or from one airway into another airway.

The inner cavity of the connection portion is provided with an occluding coating, which is provided in the inner cavity of the connection portion in a direction parallel to the radial direction of the occluding stent, to prevent food or foreign objects from passing through the inner cavity of the connection portion to enter from one alimentary canal into another alimentary canal, from one airway into one alimentary canal, from one alimentary canal into one airway, or from one airway into another airway.

The alimentary canal may be an esophagus and the airway may be a trachea.

The connection portion of the occluding stent may be tubular, the connection portion may have a diameter D1 ranging from greater than 0 and less than or equal to 10 mm, or the connection portion may have a diameter D1 ranging from greater than or equal to 9 and less than or equal to 24 mm.

The braided structure and form of the distal flange occluding body, the proximal flange occluding body, and the connection portion are configured as needs as long as the smooth compression and unfolding of the occluding stent can be achieved.

Where a surface of the connection portion is provided with a coating in itself.

Where the distal flange occluding body is umbrella-shaped, the proximal flange occluding body is mushroom cap-shaped, the connection portion is middle stent, the umbrella-shaped and the mushroom cap-shaped are connected as a whole through a middle stent. The umbrella-shaped stent and the mushroom-shaped stent are connected integrally through the middle stent. External surfaces of the umbrella-shaped stent, the mushroom-shaped stent and the middle stent are all provided with a coating. The middle stent is of a tubular structure, with both ends being sealed through the coating. The umbrella-shaped stent shapes like an umbrella when being unfolded, and the mushroom-shaped stent shapes like a pileus when being unfolded.

The umbrella-shaped stent includes an umbrella-shaped stent skeleton and an umbrella-shaped stent coating. The umbrella-shaped stent skeleton is of a dispersed structure or a braided structure. The umbrella-shaped stent coating covers the surface of the umbrella-shaped stent skeleton.

The mushroom-shaped stent includes a mushroom-shaped stent radial skeleton and a mushroom-shaped stent coating. The mushroom-shaped stent coating is provided on the surface of the mushroom-shaped stent radial skeleton. The mushroom-shaped stent radial skeleton includes a mushroom-shaped stent longitudinal skeleton, a mushroom-shaped stent proximal skeleton, and a mushroom-shaped stent distal skeleton. Similarly, the mushroom-shaped stent skeleton is of a dispersed structure or a braided structure.

The middle stent includes a middle stent skeleton and a middle stent coating. Two ends of the middle stent skeleton are integrally connected with the umbrella-shaped stent and the mushroom-shaped stent respectively. A middle stent coating is provided on the surface of the middle stent skeleton.

An esophagus-end occluding coating is provided at one end of the middle stent, and a trachea-end occluding coating is provided at the other end of the middle stent.

The umbrella-shaped stent is formed by a metal wire structure. The metal wire is made of any one or more of the following materials: a biologically compatible nickel-titanium alloy, a bioresorbable polymer, a shape-memory polymer, a resorbable metal, and a biologically compatible metal.

The mushroom-shaped stent is formed by a metal wire structure. The metal wire is made of any one or more of the following materials: a biologically compatible nickel-titanium alloy, a bioresorbable polymer, a shape-memory polymer, a resorbable metal, and a biologically compatible metal.

The middle stent is formed by a metal wire structure. The metal wire is made of any one or more of the following materials: a biologically compatible nickel-titanium alloy, a bioresorbable polymer, a shape-memory polymer, a resorbable metal, and a biologically compatible metal. A corrosion-resistant coating is provided on an external surface of the metal wire, and the corrosion-resistant coating is made of silicone. An antibacterial coating is made of an organosilicon cation-containing steroid, minocycline, rifampicin, gentamicin, vancomycin, or a hydrophobic material, or a combination thereof.

An implanter for an occluding stent is provided, where the implanter includes: an outer tube, a middle tube, a front handle, and a rear handle, the occluding stent is loaded into a distal end of the outer tube, a proximal end of the outer tube is connected to the front handle, the middle tube is loaded in the outer tube, the middle tube is connected to the rear handle, and the front handle and the outer tube are slidable relative to the rear handle and the middle tube, a pushrod is fixedly mounted in the middle tube, and the pushrod extends beyond a distal end of the middle tube; the occluding stent is loaded into a distal end of the outer tube, and the occluding stent abuts against a distal end of the pushrod.

Where the implanter is configured to release the distal flange occluding body of the occluding stent from the outer tube to unfold the distal flange occluding body into a distal tissue, and when the implanter is moved backward into a proximal tissue, release the proximal flange occluding body of the occluding stent from the outer tube to unfold the proximal flange occluding body.

Where the releasing and unfolding of the distal flange occluding body of the occluding stent from the outer tube is achieved by: pulling the front handle towards the proximal end to cause the outer tube to be moved backward together such that the distal flange occluding body of the occluding stent is exposed from the outer tube to be released and unfolded; or pushing the rear handle forward toward the distal end such that the distal flange occluding body of the occluding stent is pushed out of the outer tube by a pusher so as to be released and unfolded.

An implanter for an occluding stent includes a rear handle, a rear pushrod, and a front handle, where a flexible tube is fixedly connected to a distal end of the front handle, a proximal end of the rear pushrod is fixedly connected to the rear handle, a distal end of the rear pushrod passes through an interior of a rear pushrod locking knob and then passes through an interior of the front handle to be connected to a front pushrod, a distal end of the front pushrod is connected to a front pusher, and the front pushrod is located in the flexible tube; the front handle is lockable to or unlockable from the rear pushrod locking knob to cause the front handle to be fixed with the rear pushrod or to cause the front handle to be slidable relative to the rear pushrod, where an occluding stent is loaded into a distal end of the flexible tube and the occluding stent abuts against the front pusher.

Where a proximal end of the rear handle is provided with a rear-handle rear cover, a rear-handle front cover is connected at a distal end of the rear handle, and the rear pushrod is fixedly connected to the rear handle through a rear pushrod holder, and the distal end of the rear pushrod is connected to the front pushrod through an inner connection hose.

The front handle is provided with a Luer taper tube in communication with an inner cavity of the front handle, the distal end of the front handle is connected to a front-handle front cover through a connecting sleeve, and a distal end of the front-handle front cover is provided with a flexible sleeve hose connecting tube; the flexible sleeve hose connecting tube is connected to the flexible tube. A distal end of the rear pushrod locking knob is provided with a tapered rubber plug, and the tapered rubber plug is in contact with a tapered inner wall inside the front handle, the tapered rubber plug is provided with a through hole for the rear pushrod to pass through, the tapered rubber plug has a tapered surface in contact with the tapered inner wall, and the tapered rubber plug is provided with a cross recess, such that when a proximal end of the front handle is threaded to the rear pushrod locking knob, the tapered rubber plug holds the rear pushrod tightly.

The implanter for an occluding stent is also provided with an inner hole for the guide wire to pass through one side of the occluding stent.

A range-adjustable mark is provided on the rear pushrod between the rear pushrod locking knob and a rear-handle front cover of the implanter, the range-adjustable mark comprises a positioning nut located at the distal end and a range adjuster that is located at the proximal end and is in thread connection with the positioning nut, the positioning nut is provided with an anti-slip rib, the range adjuster is provided with an alary protrusion, the positioning nut and the range adjuster are sleeved over the rear pushrod, and the range adjuster is screwed in or out to adjust a distance between the rear pushrod locking knob and the rear-handle front cover.

A removable mark is provided on the rear pushrod between the rear pushrod locking knob and a rear-handle front cover of the implanter, the removable mark is sleeved over the rear pushrod and comprises a removable sleeve and an anti-slip handle, the removable sleeve and the anti-slip handle are connected through a connector, a notch for allowing the rear pushrod to enter is provided on a side of the removable sleeve, the removable mark is sleeved over the rear pushrod through the notch, and a connection is implemented replying on an elastic force of the removable sleeve, so that a distance between the rear pushrod locking knob and the rear-handle front cover is locked.

In one embodiment, a method for implanting an implanter loading an occluding stent, including the steps of:
locating a position of a fistula, where the fistula is located between a first body channel and a second body channel;
feeding a distal end of the implanter to pass from a first side of the fistula in the first body channel through the fistula to a second side of the fistula in the second body channel, where the occluding stent is loaded into the flexible tube at the distal end of the implanter;
releasing the distal flange occluding body of the occluding stent from the distal end of the flexible tube to unfold the distal flange occluding body of the occluding stent at the second side of the fistula in the second body channel;
moving the implanter backward towards the proximal end to make the distal end of the flexible tube return to the first side of the fistula in the first body channel, and releasing the proximal flange occluding body of the occluding stent from the distal end of the flexible tube to unfold the proximal flange occluding body of the occluding stent at the first side of the fistula in the first body channel; and
withdrawing the implanter to an outside of a body.

In one embodiment, the first body channel is an alimentary canal and the second body channel is an airway. Specifically, the first body channel can be an esophagus, the second body channel can be a trachea.

In one embodiment, a method for implanting an implanter loading an occluding stent, including the steps of:
when the fistula has a size of less than 9 mm, the method comprises steps of:
feeding a first endoscope having a clamp channel of great than or equal to 3.2 mm into the first body channel, and feeding a second endoscope into the second body channel, and locating the position of the fistula in combination with the first endoscope and the second endoscope, the fistula is located between the first body channel and the second body channel;
feeding the implanter through the clamp channel of the first endoscope to allow the distal end of the implanter to pass through the fistula from the first side of the fistula in the first body channel to the second side of the fistula in the second body channel under the supervision of the first endoscope, and confirming that the distal end of the implanter has entered the second side of the fistula under the supervision of the second endoscope, wherein the occluding stent is loaded in the flexible tube at the distal end of the implanter and abuts against the front pusher, and the connection portion of the occluding stent has a diameter D1 of 2 mm to 9 mm;
unlocking the front handle of the implanter from the rear actuator locking knob of the implanter to allow the front handle to be slidable relative to the rear pushrod, and pulling the front handle of the implanter backward towards the proximal end under the supervision of the second endoscope to allow the flexible tube to be moved backward together, so that the distal flange occluding body of the occluding stent is exposed and released from the flexible tube and is unfolded at the second side of the fistula in the second body channel; or pushing the rear handle forward towards the distal end to cause the front pusher to push the distal flange occluding body of the occluding stent out of the flexible tube, so that the distal flange occluding body of the occluding stent is released and unfolded at the second side of the fistula in the second body channel;
slowly moving the implanter backward towards the proximal end until the distal end of the flexible tube of the implanter is observed under the first endoscope to have returned to the first side of the fistula in the first body channel, pulling the front handle of the implanter backward towards the proximal end to cause the flexible tube to be moved backward together, so that the proximal flange occluding body of the occluding stent is exposed and released from the flexible tube and is unfolded at the first side of the fistula in the first body channel; or locking the front handle to the rear pushrod locking knob to cause the rear pushrod to be fixed to the front handle, and moving the implanter backward towards the proximal end, so that the distal flange occluding body is blocked by an inner wall of the second human second channel where the second side of the fistula is located and thus the proximal flange occluding body of the occluding stent is pulled out of the flexible tube and released and unfolded at the first side of the fistula in the first body channel; and
withdrawing the implanter to the outside of the body.

In this embodiment, the first endoscope is a gastroscope or a colonoscope, and the second endoscope is a tracheoscope. The first body channel is an alimentary canal and the second body channel is an airway. Specifically, the first body channel can be an esophagus, the second body channel can be a trachea.

In one embodiment, a method for implanting an implanter loading an occluding stent, including the steps of:
when the fistula has a size of greater than 9 mm, the method comprises steps of:
feeding a first endoscope and an over tube into the first body channel, wherein the first endoscope is placed inside the over tube, and feeding a second endoscope into a second body channel, and locating the position of the fistula in combination with the first endoscope and the second endoscope, wherein the fistula is located between the first body channel and the second body channel;
feeding a distal end of the first endoscope to pass through the fistula from the first side of the fistula in the first body channel to the second side of the fistula in the second body channel, feeding a distal end of the over tube along the first endoscope to the second side of the fistula in the second body channel under the supervision of the second endoscope, and then withdrawing the first endoscope from the over tube to the outside of the body;
feeding a distal end of the implanter through an inner lumen of the over tube to the second side of the fistula in the second body channel under the supervision of the second endoscope, leaving the distal end of the implanter at the second side of the fistula in the second body channel, and moving the over tube backward to the first side of the fistula in the first body channel, wherein the occluding stent is loaded in the flexible tube at the distal end of the implanter and abuts against the front pusher, and the connection portion of the occluding stent has a diameter D1 of 9 mm to 24 mm;
feeding the first endoscope along an outer side of the over tube to the first side of the fistula in the first body channel to observe the placement of the implanter in the first body channel; unlocking the front handle of the implanter from the rear pushrod locking knob of the implanter to allow the front handle to be slidable relative to the rear pushrod, and pulling the front handle of the implanter backward towards the proximal end under the supervision of the second endoscope to allow the flexible tube to be moved backward together, so that the distal flange occluding body of the occluding stent is exposed and released from the flexible tube and is unfolded at the second side of the fistula in the second body channel; or pushing the rear handle forward towards the distal end to cause the front pusher to push the distal flange occluding body of the occluding stent out of the flexible tube, so that the distal flange occluding body of the occluding stent is released and unfolded at the second side of the fistula in the second body channel;
moving the over tube backward towards the proximal end to the flexible tube of the implanter to fully expose the distal end of the implanter, slowly moving the implanter backward towards the proximal end until the distal end of the flexible tube of the implanter is observed under the first endoscope to have returned to the first side of the fistula in the first body channel, pulling the front handle of the implanter backward towards the proximal end to cause the flexible tube to be moved backward together, so that the proximal flange occluding body of the occluding stent is exposed and released from the flexible tube and is unfolded at the first side of the fistula in the first body channel under the supervision of the first endoscope; or locking the front handle to the rear pushrod locking knob to cause the rear pushrod to be fixed to the front handle, and moving the implanter backward towards the proximal end, so that the distal flange occluding body is blocked by an inner wall of the second human second channel where the second side of the fistula is located and thus the proximal flange occluding body of the occluding stent is pulled out of the flexible tube and released and unfolded at the first side of the fistula in the first body channel; and withdrawing the implanter to the outside of the body.

In this embodiment, the first endoscope is an ultrafine gastroscope, and the second endoscope is a tracheoscope. The first body channel is an alimentary canal and the second body channel is an airway. Specifically, the first body channel can be an esophagus, the second body channel can be a trachea.

In one embodiment, a method for implanting an implanter loading an occluding stent, including the steps of:
locating the position of the fistula under an endoscope, where the fistula is located between the first body channel and the second body channel;
feeding the implanter through a clamp channel of the endoscope to allow the distal end of the implanter to pass through the fistula from the first side of the fistula in the first body channel to the second side of the fistula in the second body channel under the supervision of the endoscope, wherein the occluding stent is loaded in the flexible tube at the distal end of the implanter;
unlocking the front handle of the implanter from the rear actuator locking knob of the implanter to allow the front handle to be slidable relative to the rear pushrod, and pulling the front handle of the implanter backward towards the proximal end under the supervision of the endoscope to allow the flexible tube to be moved backward together, so that the distal flange occluding body of the occluding stent is exposed and released from the flexible tube and is unfolded at the second side of the fistula in the second body channel; or pushing the rear handle forward towards the distal end to cause the front pusher to push the distal flange occluding body of the occluding stent out of the flexible tube, so that the distal flange occluding body of the occluding stent is released and unfolded at the second side of the fistula in the second body channel;
slowly moving the implanter backward towards the proximal end until the distal end of the flexible tube of the implanter is observed under the endoscope to have returned to the first side of the fistula in the first body channel, pulling the front handle of the implanter backward towards the proximal end to cause the flexible tube to be moved backward together, so that the proximal flange occluding body of the occluding stent is exposed and released from the flexible tube and is unfolded at the first side of the fistula in the first body channel; or locking the front handle to the rear pushrod locking knob lock to cause the rear pushrod to be fixed to the front handle, and moving the implanter backward towards the proximal end, so that the distal flange occluding body is blocked by an inner wall of the second human second channel where the second side of the fistula is located and thus the proximal flange occluding body of the occluding stent is pulled out of the flexible tube and released and unfolded at the first side of the fistula in the first body channel.
withdrawing the implanter to the outside of the body.

In this embodiment, the endoscope is a gastroscope. The first body channel is an alimentary canal and the second body channel is an airway. Specifically, the first body channel can be an esophagus, the second body channel can be a trachea.

In one embodiment, in a method for implanting an implanter loading an occluding stent further includes, after the occluding stent is released and the implanter is withdrawn, the method further includes the step of testing whether the occluding stent completely closes the fistula, including: injecting a methylene blue mucosal stain to the first side of the fistula, confirming whether the methylene blue mucosal stain is leaked to the second side of the fistula so as to confirm whether the occluding stent completely closes the fistula. In one embodiment, the methylene blue mucosal stain is injected to the esophageal side through a clamp channel of a gastroscope or enteroscope, then whether the methylene blue mucosal stain is leaked to the tracheal side is confirmed by a tracheoscope, and if there is no leakage the endoscope is withdrawn.

In one embodiment, a method for implanting an implanter loading an occluding stent further includes after the proximal flange occluding body of the occluding stent is unfolded, confirming, by an endoscope, whether the proximal flange occluding body of the occluding stent does not fall off, and then withdrawing the implanting.

In one embodiment, the fistula may be an esophagobronchial fistula.

Compared with the prior art, the present application has the following beneficial effects:
In the present application, because the distal end and the proximal end of the occluding stent are single-layer structures, the occluding stent is lighter than a disc-shaped structure of a vascular occluder, and surrounding tissues of the fistula have a lower necrosis risk. In addition, the esophagus end is larger than the trachea end, and the risk of the occluder falling into the trachea is further reduced. The stent has no end cover. Even if the occluder is deformed to be pear-shaped, it does not touch the surrounding tissues. Moreover, the occluding stent is released under the direct vision of the gastroscope, so that operations of the doctor are more reliable. The stent is provided with a withdraw line at the proximal end, and can be taken out at any time through conservative medical therapy. Even if falling into the trachea, the stent can be taken out by using foreign body forceps. The present application substitutes the treatment method of expanding the esophagus circumferentially by using a conventional stent, the esophagus is not compressed, thereby alleviating the pain and improving the life quality of the patient. The stent is lighter than an occluder and has smaller compression on surrounding tissues, so that the surrounding tissues have a lower risk of avascular necrosis.

### BRIEF DESCRIPTION OF DRAWINGS

FIG. 1 is a schematic structural diagram of Embodiment 1 of a stent according to the present application.

FIG. 2 is a side view of Embodiment 1 of a stent according to the present application.

FIG. 3 is a three-dimensional schematic structural diagram of Embodiment 1 of a stent according to the present application.

FIG. 4 is a three-dimensional schematic structural diagram of Embodiment 1 of a stent according to the present application.

FIG. 5 is a schematic structural diagram of Embodiment 2 of a stent according to the present application.

FIG. 6 is a three-dimensional schematic structural diagram of Embodiment 2 of a stent according to the present application.

FIG. 7 is a cross-sectional schematic structural diagram of a stent located in an implanter according to the present application.

FIG. 8 is a cross-sectional schematic structural diagram of a partial unfolded state of a stent partially located in an implanter according to the present application.

FIG. 9a is a first sectional schematic structural diagram of an implanter according to the present application.

FIG. 9b is a second sectional schematic structural diagram of the implanter according to the present application.

FIG. 10 is an exploded schematic structural diagram of the implanter according to the present application.

FIG. 11 is a schematic structural diagram of overall assembly of the implanter according to the present application.

FIG. 12 is a schematic diagram of a first breakdown step of implanting a stent into an esophagobronchial fistula by using an implanter according to the present application.

FIG. 13 is a schematic diagram of a second breakdown step of implanting the stent into the esophagobronchial fistula by using the implanter according to the present application.

FIG. 14 is a schematic diagram of a third breakdown step of implanting the stent into the esophagobronchial fistula by using the implanter according to the present application.

FIG. 15 is a first schematic structural diagram of a tapered rubber plug according to the present application.

FIG. 16 is a second schematic structural diagram of the tapered rubber plug according to the present application.

FIG. 17 is a third schematic structural diagram of the tapered rubber plug according to the present application.

FIG. 18 is a fourth schematic structural diagram of the tapered rubber plug according to the present application.

FIG. 19 is a schematic structural diagram of another occluding stent according to the present application.

FIG. 20 is a schematic structural diagram of a first target and a second target according to the present application.

FIG. 21 is a schematic structural diagram of Embodiment 8 of the present application.

FIG. 22 is a side schematic structural diagram of an occluding stent in Embodiment 9 of the present application.

FIG. 23 is an overall schematic structural diagram of the occluding stent in Embodiment 9 of the present application.

FIG. 24 is a first three-dimensional schematic diagram of the occluding stent in Embodiment 9 of the present application.

FIG. 25 is a second three-dimensional schematic diagram of the occluding stent in Embodiment 9 of the present application.

FIG. 26 is a side schematic structural diagram of an occluding stent in Embodiment 10 of the present application.

FIG. 27 is an overall schematic structural diagram of the occluding stent in Embodiment 10 of the present application.

FIG. 28 is a three-dimensional schematic diagram of the occluding stent in Embodiment 10 of the present application.

FIG. 29 is a side schematic structural diagram of an occluding stent in Embodiment 11 of the present application.

FIG. 30 is an overall schematic structural diagram of the occluding stent in Embodiment 11 of the present application.

FIG. 31 is a first three-dimensional schematic diagram of the occluding stent in Embodiment 11 of the present application.

FIG. 32 is a second three-dimensional schematic diagram of the occluding stent in Embodiment 11 of the present application.

FIG. 33 is a side schematic structural diagram of an occluding stent in Embodiment 12 of the present application.

FIG. 34 is an overall schematic structural diagram of the occluding stent in Embodiment 12 of the present application.

FIG. 35 is a first three-dimensional schematic diagram of the occluding stent in Embodiment 12 of the present application.

FIG. 36 is a second three-dimensional schematic diagram of the occluding stent in Embodiment 12 of the present application.

FIG. 37 is a side schematic structural diagram of an occluding stent in Embodiment 13 of the present application.

FIG. 38 is an overall schematic structural diagram of the occluding stent in Embodiment 13 of the present application.

FIG. 39 is a first three-dimensional schematic diagram of the occluding stent in Embodiment 13 of the present application.

FIG. 40 is a second three-dimensional schematic diagram of the occluding stent in Embodiment 13 of the present application.

FIG. 41 is a side schematic structural diagram of an occluding stent in Embodiment 14 of the present application.

FIG. 42 is an overall schematic structural diagram of the occluding stent in Embodiment 14 of the present application.

FIG. 43 is a first three-dimensional schematic diagram of the occluding stent in Embodiment 14 of the present application.

FIG. 44 is a second three-dimensional schematic diagram of the occluding stent in Embodiment 14 of the present application.

FIG. 45 is a side schematic structural diagram of an occluding stent in Embodiment 15 of the present application.

FIG. 46 is an overall schematic structural diagram of the occluding stent in Embodiment 15 of the present application.

FIG. 47 is a first three-dimensional schematic diagram of the occluding stent in Embodiment 15 of the present application.

FIG. 48 is a second three-dimensional schematic diagram of the occluding stent in Embodiment 15 of the present application.

FIG. 49 is a partial schematic diagram of a connection portion of the occluding stent in Embodiment 15 of the present application.

FIG. 50 is a schematic structural diagram of a range-adjustable mark in Embodiment 16 of the present application.

FIG. 51 is a schematic structural diagram of a removable mark in Embodiment 17 of the present application.

FIG. 52 is a flowchart of a method for implanting an implanter including an occluding stent according to some embodiments of the present application.

FIG. 53 is a flowchart of a method for implanting an implanter including an occluding stent according to some embodiments of the present application.

FIG. 54 is a schematic diagram of a structure of an over tube according to some embodiments of the present application.

FIG. 55 is a cross-sectional view of the over tube shown in FIG. 54.

FIG. 56 is a schematic diagram of an overall assembly structure of an implanter according to one embodiment of the present application.

FIG. 57 is a schematic diagram of a cross-sectional structure of the implanter of FIG. 56 of the present application.

FIG. 58 shows a partially enlarged view of a front portion of an implanter of the present application.

In the drawings: 1, umbrella-shaped stent; 100, umbrella-shaped stent coating; 101, umbrella-shaped stent skeleton; 2, mushroom-shaped stent; 20, mushroom-shaped stent coating; 21, mushroom-shaped stent radial skeleton; 23, mushroom-shaped stent longitudinal skeleton; 24, mushroom-shaped stent proximal skeleton; 25, mushroom-shaped stent distal skeleton; 3, middle stent; 30, middle stent coating; 31, middle stent skeleton; 4, esophagus-end occluding coating; 5, trachea-end occluding coating; 7, withdraw line; 8, 0140, rear handle; 80, 0200, rear-handle rear cover; 81, 0130, rear-handle front cover; 9, rear pushrod; 90, rear pushrod holder; 91, inner connection hose; 92, front pushrod; 93, 0010, front pusher; 94, flexible tube; 95, precise locating sleeve; 110, rear pushrod locking knob; 111, tapered inner wall; 112, Luer taper tube; 113, tapered rubber plug; 114, inner cavity; 115, 0090, front handle; 116, 0080, connecting sleeve; 117, 0070, front-handle front cover; 118, sleeve hose connecting tube; 1131, through hole; 1132, tapered surface; 1133, cross recess; 1A, mirror umbrella-shaped stent; 119, first target; 120, second target; 6, occluding stent; 60, distal flange occluding body; 61, proximal flange occluding body; 62, connection portion; 63, first occluding coating; 64, second occluding coating; 65, first occluding portion end; 66, second occluding end; 801, removable sleeve; 802, anti-slip handle; 803, notch; 804, connector; 805, positioning nut; 806, range adjuster; 807, alary protrusion; 808, anti-slip rib; 1000, implanter; 601, first shoulder; 611, second shoulder; 65′, 66′ end surface; 0060, outer tube; 0050, middle tube; 0030, developer ring; 0040, inner tube; 0020, inner core; 0120, middle tube locking knob; 0110, locking ring; 0111, inner wall; 0100, sealing ring; 0150, Luer taper; 0210, tail cover cap; 0170, fixing ring; 0300, medical grade high clarity PVC; 0400, stainless steel spring; 0500, ABS handle; 0600, black printed mark

### DESCRIPTION OF EMBODIMENTS

The technical solutions in the embodiments of the present application are described clearly and completely below with reference to the accompanying drawings in the embodiments of the present application. Apparently, the described embodiments are merely some embodiments, rather than all embodiments, of the present application. All other embodiments obtained by a person of ordinary skill in the art based on the embodiments in the present application without creative efforts belong to the protection scope of the present application.

In the description of the present application, it should be appreciated that the directions or positional relations indicated by the terms such as “center”, “longitudinal”, “lateral”, “length”, “width”, “thickness”, “upper”, “lower”, “front”, “rear”, “left”, “right”, “vertical”, “horizontal”, “top”, “bottom”, “inner”, “outer”, “clockwise” and “anticlockwise” are directions or positional relations based on the drawings, and are merely used for describing the present application and simplifying the description, rather than indicating or implying that a described device or element needs to have a specific direction or be constructed and operated in a specific direction. Therefore, such terms cannot be construed as a limitation on the present application.

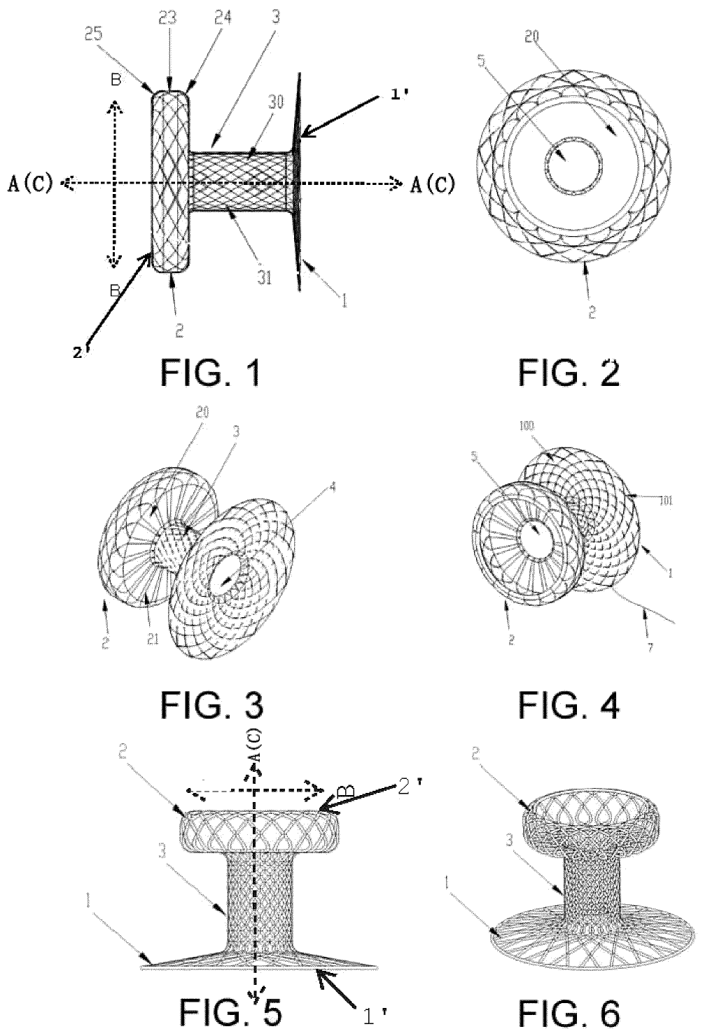

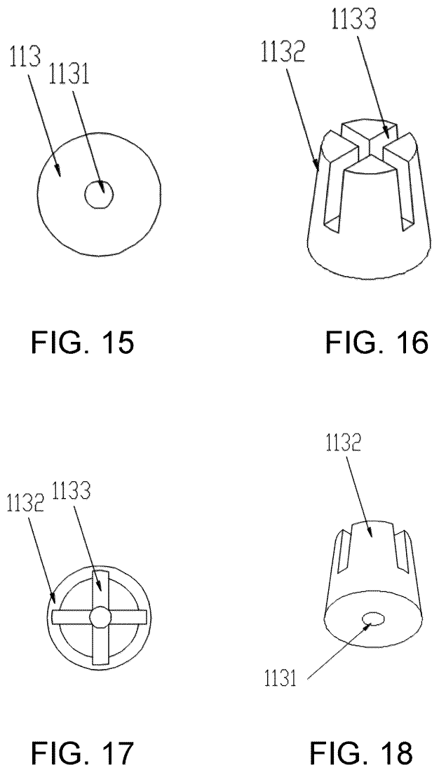

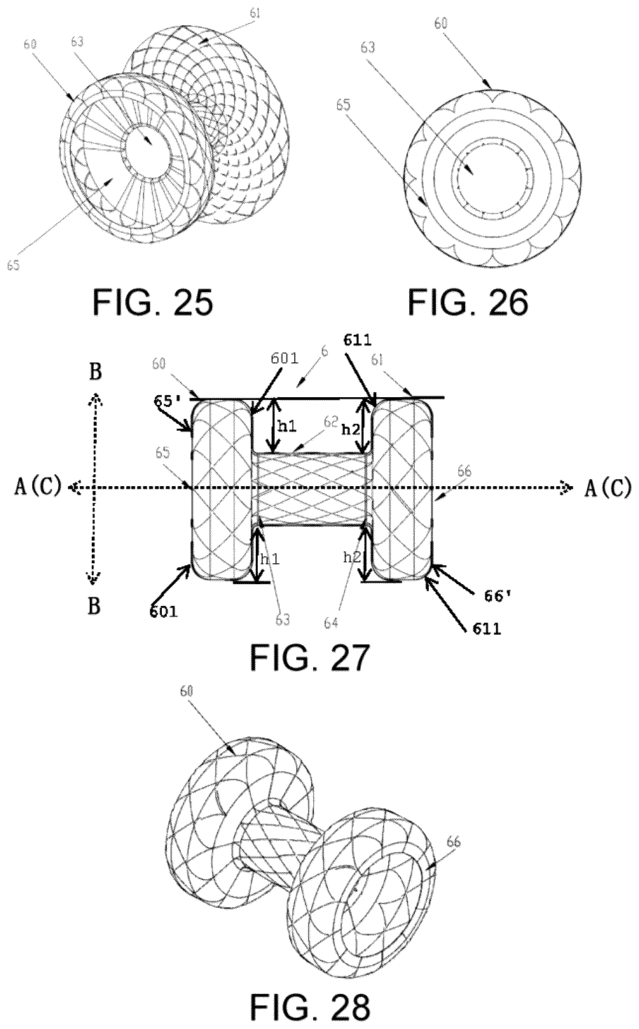

In FIG. 1 to FIG. 58 and the descriptions throughout the present application, the term “proximal end” represents an end close to an operator, and the term “distal end” represents an end away from the operator.

The term “longitudinal direction A” in these instructions is based on the blocking bracket, as shown in all figures. The term “radial direction B” in this specification is based on the blocking bracket, as shown in all figures.

The term “longitudinal direction A” in the present application is based on occluding stents, as shown in all figures. The term “radial direction B” in the present application is based on occluding stents, as shown in all figures.

According to all figures, the “vertical” direction is the radial direction B, and the “horizontal” and “longitudinal” directions can be the longitudinal direction A. “front” and “left” directions may be the same relative to the longitudinal direction A of the occluding stent as illustrated in FIGS. 1, 19, 23, 27, 30, 34, 38, 42, 46, and 63, and “rear” and “right” directions can be the same relative to the longitudinal direction A of the occluding stent as illustrated in FIGS. 1, 19, 23, 27, 30, 34, 38, 42, 43, and 64. “up” and “down” directions may be the “top” and “bottom” of the occluding stent, as shown in the radial direction B in FIGS. 1, 19, 23, 27, 30, 34, 38, 42, 46, and 63. The “transverse”, “length”, “width”, and “thickness” directions can be either the longitudinal direction A or radial direction B of the occluding stent, as shown in FIGS. 1, 19, 23, 27, 30, 34, 38, 42, 46, and 63.

### Embodiment 1

As shown in FIG. 1 to FIG. 4, characterized in that, an occluding stent includes an umbrella-shaped stent 1, a mushroom-shaped stent 2, and a middle stent 3. The umbrella-shaped stent 1 and the mushroom-shaped stent 2 are connected integrally through the middle stent 3. External surfaces of the umbrella-shaped stent 1, the mushroom-shaped stent 2 and the middle stent 3 are all provided with a coating. The middle stent 3 is of a tubular structure, with both ends being sealed through the coating. The umbrella-shaped stent 1 shapes like an umbrella when being unfolded, and the mushroom-shaped stent 2 shapes like a pileus when being unfolded.

The umbrella-shaped stent 1 includes an umbrella-shaped stent skeleton 101 and an umbrella-shaped stent coating 100. The umbrella-shaped stent skeleton 101 is of a dispersed structure or a braided structure. The umbrella-shaped stent coating 100 covers the surface of the umbrella-shaped stent skeleton 101.

The mushroom-shaped stent 2 includes a mushroom-shaped stent radial skeleton 21 and a mushroom-shaped stent coating 20. The mushroom-shaped stent coating 20 is provided on the surface of the mushroom-shaped stent radial skeleton 21. The mushroom-shaped stent radial skeleton 21 includes a mushroom-shaped stent longitudinal skeleton 23, a mushroom-shaped stent proximal skeleton 24, and a mushroom-shaped stent distal skeleton 25.

The middle stent 3 includes a middle stent skeleton 31 and a middle stent coating 30. Two ends of the middle stent skeleton 31 are integrally connected with the umbrella-shaped stent 1 and the mushroom-shaped stent 2 respectively. A middle stent coating 30 is provided on the surface of the middle stent skeleton 31.

An esophagus-end occluding coating 4 is provided at one end of the middle stent 3, and a trachea-end occluding coating 5 is provided at the other end of the middle stent 3.

The umbrella-shaped stent 1 is formed by a metal wire structure. The metal wire is made of any one or more of the following materials: a biologically compatible nickel-titanium alloy, a bioresorbable polymer, a shape-memory polymer, a resorbable metal, and a biologically compatible metal.

The mushroom-shaped stent 2 is formed by a metal wire structure. The metal wire is made of any one or more of the following materials: a biologically compatible nickel-titanium alloy, a bioresorbable polymer, a shape-memory polymer, a resorbable metal, and a biologically compatible metal.

The middle stent 3 is formed by a metal wire structure. The metal wire is made of any one or more of the following materials: a biologically compatible nickel-titanium alloy, a bioresorbable polymer, a shape-memory polymer, a resorbable metal, and a biologically compatible metal.

A corrosion-resistant coating is provided on an external surface of the metal wire, and the corrosion-resistant coating is made of silicone. An antibacterial coating is made of an organosilicon cation-containing steroid, minocycline, rifampicin, gentamicin, vancomycin, or a hydrophobic material, or a combination thereof.

A withdraw line 7 is connected on the umbrella-shaped stent 1. The withdraw line 7 may be a metal wire made of the material mentioned above, or a non-metal material with good biological compatibility.

In the longitudinal direction A of the occluding stent, the end surface 1′ of the outermost end of the umbrella-shaped stent 1 is flat when viewed from the radial direction B of the occluding stent, and has no protrusion outwardly in the longitudinal direction A away from the middle stent 3. The end surface 2′ of the outermost end of the mushroom-shaped stent 2 is flat when viewed from the radial direction B of the occluding stent and has no protrusion outwardly in the longitudinal direction A away from the middle stent 3. Where the radial direction B is the radial direction of the umbrella-shaped stent 1 and the mushroom-shaped stent 2, and the longitudinal direction A is perpendicular to the radial direction B.

The end surface 1′ of the outermost end of the umbrella-shaped stent 1 and the end surface 2′ of the outermost end of the mushroom-shaped stent 2 are in the shape having an opening in their middle portions, and an edge of the opening is at a certain distance from a center axis C in the longitudinal direction A of the occluding stent. It can be seen in the figure that the entire end surfaces 1 ‘ and 2’ are not completely braided by metal wires and the middle portions of the end surfaces are empty and not braided. The dotted line representing the longitudinal direction A of the occluding stent can also represent the center axis C in the longitudinal direction A of the occluding stent, as can be seen from FIG. 1.

Thus, the proximal and distal ends of the occluding stent do not have end covers or tails protruding outwardly, thereby preventing damage to surrounding tissues.

### Embodiment 2

As shown in FIG. 5 and FIG. 6, an occluding stent is provided, where the metal wire adopts a braided structure and form that are different from those in Embodiment 1, characterized in that, the occluding stent includes an umbrella-shaped stent 1, a mushroom-shaped stent 2, and a middle stent 3. The umbrella-shaped stent 1 and the mushroom-shaped stent 2 are connected integrally through the middle stent 3. External surfaces of the umbrella-shaped stent 1, the mushroom-shaped stent 2 and the middle stent 3 are all provided with a coating. The middle stent 3 is of a tubular structure, with both ends being sealed through the coating. The umbrella-shaped stent 1 shapes like an umbrella when being unfolded, and the mushroom-shaped stent 2 shapes like a pileus when being unfolded.

In the longitudinal direction A of the occluding stent, the end surface 1′ of the outermost end of the umbrella-shaped stent 1 is flat when viewed from the radial direction B of the occluding stent, and has no protrusion outwardly in the longitudinal direction A away from the middle stent 3. The end surface 2′ of the outermost end of the mushroom-shaped stent 2 is flat when viewed from the radial direction B of the occluding stent and has no protrusion outwardly in the longitudinal direction A away from the middle stent 3. Where the radial direction B is the radial direction of the umbrella-shaped stent 1 and the mushroom-shaped stent 2, and the longitudinal direction A is perpendicular to the radial direction B.

The end surface 1′ of the outermost end of the umbrella-shaped stent 1 and the end surface 2′ of the outermost end of the mushroom-shaped stent 2 are in the shape having an opening in their middle portions, and an edge of the opening is at a certain distance from a center axis C in the longitudinal direction A of the occluding stent. It can be seen in the figure that the entire end surfaces 1′ and 2′ are not completely braided by metal wires and the middle portions of the end surfaces are empty and not braided. The dotted line representing the longitudinal direction A of the occluding stent can also represent the center axis C in the longitudinal direction A of the occluding stent, as can be seen from FIG. 1.

Thus, the proximal and distal ends of the occluding stent do not have end covers or tails protruding outwardly, thereby preventing damage to surrounding tissues.

### Embodiment 3

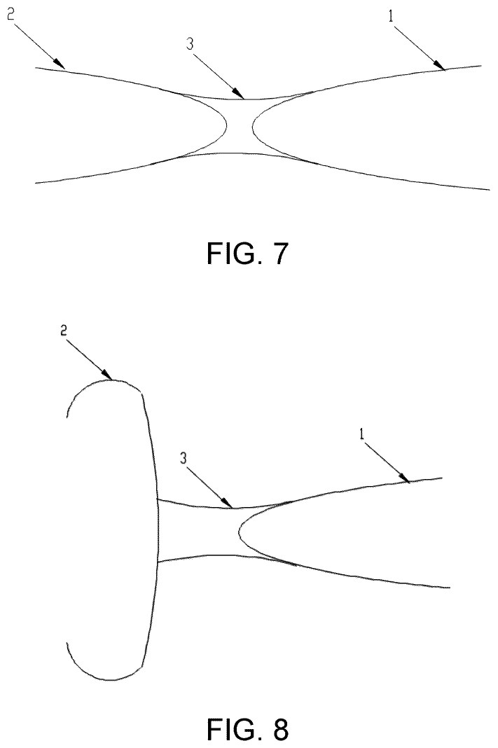

FIG. 7 is a cross-sectional schematic structural diagram of a stent located in an implanter according to the present application. The whole stent is compressed and folded in the tubular structure of the implanter, and is delivered to a target esophagobronchial fistula area.

FIG. 8 is a cross-sectional schematic structural diagram of a partial unfolded state of a stent partially located in an implanter according to the present application. An end of the stent which is located at the esophagus side or the trachea side is pushed out by the implanter, and is unfolded under the effect of its own elastic force and a memory tension. Then, during a retraction process of the implanter, the other end of the stent is pushed out, released and unfolded, to complete closure of the esophagobronchial fistula.

### Embodiment 4

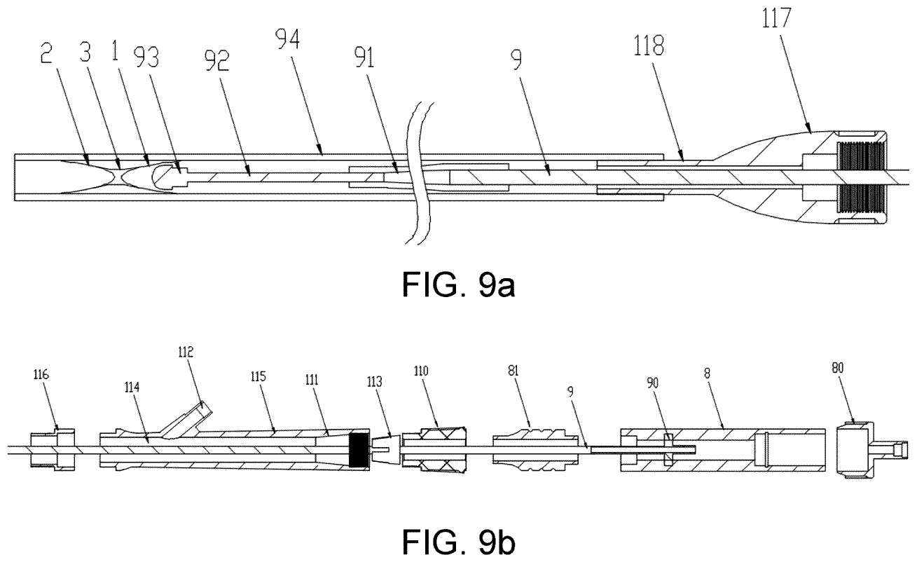

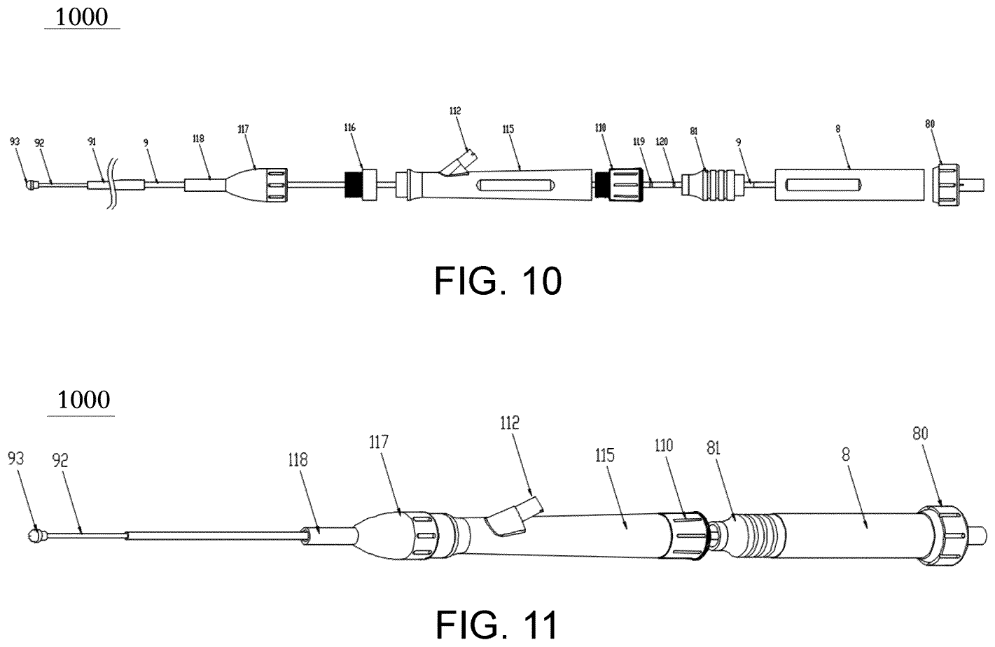

FIG. 9a is a first sectional schematic structural diagram of an implanter according to the present application. FIG. 9b is a second sectional schematic structural diagram of the implanter according to the present application. FIG. 10 is an exploded schematic structural diagram of the implanter according to the present application. FIG. 11 is a schematic structural diagram of overall assembly of the implanter according to the present application.

An occluding stent implanter includes a rear handle 8, a rear pushrod 9 and a front handle 115. The rear pushrod 9 is fixedly connected to the rear handle 8. The rear pushrod 9 passes through an interior of a rear pushrod locking knob 110 and then passes through the interior of the front handle 115. A distal end of the rear pushrod 9 is connected to a front pushrod 92 through an inner connection hose 91. A mushroom-shaped front pusher 93 is connected at the distal end of the front pushrod 92. A flexible tube 94 is sleeved over the front pushrod 92. The occluding stent is loaded into the flexible tube 94 and abuts against the front pusher 93. A proximal end of the front handle 115 is connected to a rear pushrod locking knob 110 through threads to lock the front handle 115 to the rear pushrod locking knob 110, so that the front handle 115 is fixedly connected to the rear pushrod locking knob 110. When the front handle 115 is released from the rear pushrod locking knob 110, so that the two are unlocked, the front handle 115 can slide relative to the rear pushrod 9. A tapered rubber plug 113 is disposed at a distal end of the rear pushrod locking knob 110. The tapered rubber plug 113 is in contact with a tapered inner wall 111 inside the front handle 115.

The rear pushrod 9 is a metal rod. The front pusher 93, the rear handle 8 and the front handle 115 may be made of plastic through molding. The inner connection hose 91 and the flexible tube 94 are hoses made of a flexible material.

A rear-handle rear cover 80 is provided at a proximal end of the rear handle 8. A rear-handle front cover 81 is connected at a distal end of the rear handle 8. The rear pushrod 9 is fixedly connected to the rear handle 8 through a rear pushrod holder 90.

The front handle 115 is provided with a Luer taper tube 112 in communication with an inner cavity 114 of the front handle 115. A distal end of the front handle 115 is connected to a front-handle front cover 117 through a connecting sleeve 116, and a flexible sleeve hose connecting tube 118 is disposed at a distal end of the front-handle front cover 117. A flexible tube 94 is connected on the sleeve hose connecting tube 118. The tapered rubber plug 113 is made of a rubber and is elastic. The tapered rubber plug 113 is provided with a through hole 1131 for the rear pushrod 9 to pass through. The tapered rubber plug 113 has a tapered surface 1132 in contact with the tapered inner wall 111, and the tapered rubber plug 113 is provided with a cross recess 1133. When the proximal end of the front handle 115 is in threaded connection with the rear pushrod locking knob 110 to lock the two, the tapered rubber plug 113 is axially squeezed, and under the effect of the tapered inner wall 111, the tapered rubber plug 113 holds the rear pushrod 9 tightly, so that the rear pushrod 9 is fixed with the front handle 115.

The Luer taper tube 112 may be connected to a syringe or the like, to wash or suction the inner cavity 114.

### Embodiment 5

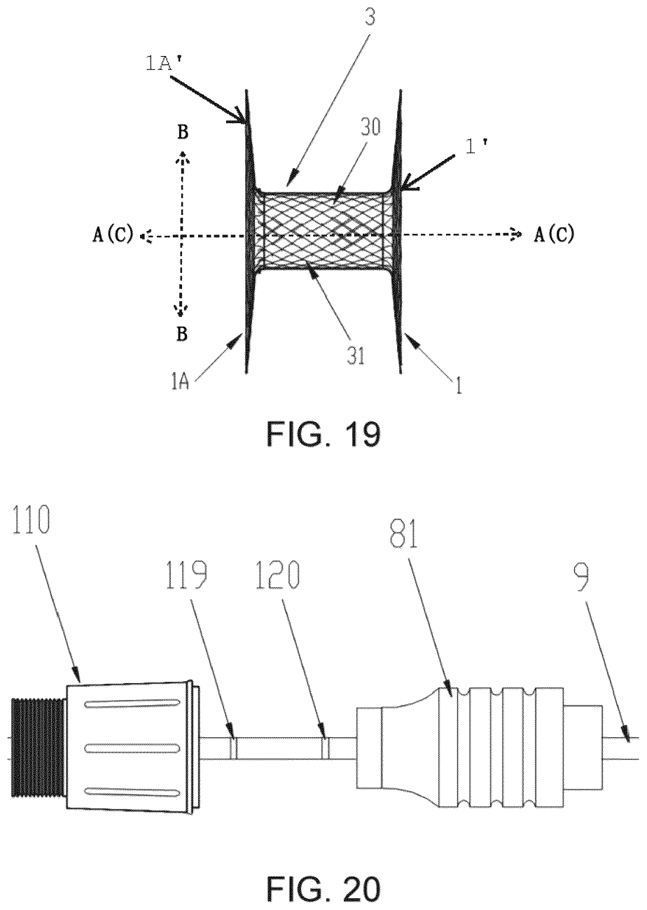

FIG. 19 is a schematic structural diagram of another occluding stent according to the present application.

The mushroom-shaped stent 2 may be replaced with a mirror umbrella-shaped stent 1A of which two ends are symmetrical about a mirror line.

In the longitudinal direction A of the occluding stent, the end surface 1′ of the outermost end of the umbrella-shaped stent 1 is flat when viewed from the radial direction B of the occluding stent, and has no protrusion outwardly in the longitudinal direction A away from the middle stent 3. The end surface 2′ of the outermost end of the umbrella-shaped stent 1A is flat when viewed from the radial direction B of the occluding stent and has no protrusion outwardly in the longitudinal direction A away from the middle stent 3. Where the radial direction B is the radial direction of the umbrella-shaped stent 1 and the umbrella-shaped stent 1A, and the longitudinal direction A is perpendicular to the radial direction B.

The end surface 1′ of the outermost end of the umbrella-shaped stent 1 and the end surface 2′ of the outermost end of the umbrella-shaped stent 1A are in the shape having an opening in their middle portions, and an edge of the opening is at a certain distance from a center axis C in the longitudinal direction A of the occluding stent. It can be seen in the FIG. that the entire end surfaces 1′ and 2′ are not completely braided by metal wires and the middle portions of the end surfaces are empty and not braided. The dotted line representing the longitudinal direction A of the occluding stent can also represent the center axis C in the longitudinal direction A of the occluding stent, as can be seen from FIG. 1.

Thus, the proximal and distal ends of the occluding stent do not have end covers or tails protruding outwardly, thereby preventing damage to surrounding tissues.

### Embodiment 6

FIG. 20 is a schematic structural diagram of a first target and a second target according to the present application. The rear pushrod 9 is further provided with a first target 119 and a second target 120. The first target 119 and the second target 120 are a yellow scale mark and a red scale mark respectively, which represent that releasing of the trachea-side stent is started and releasing of the trachea-side stent is completed respectively.

### Embodiment 7

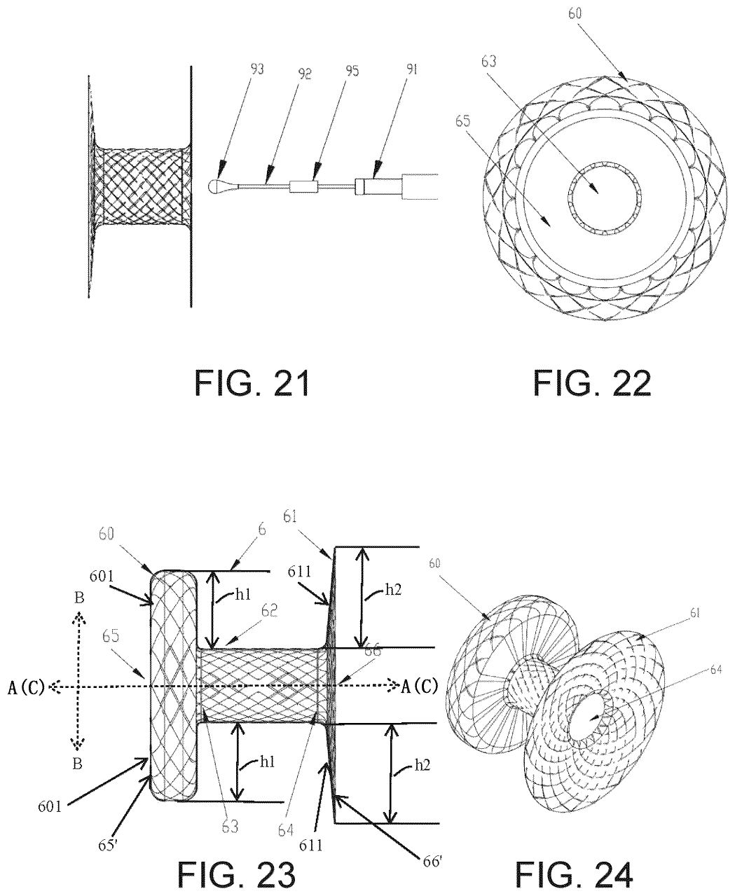

As shown in FIG. 21, the distal end of the front pusher 93 is rounded, to better push out the stent without damaging the occluding coating; the proximal end of the front pusher 93 is teardrop-shaped, so that it is more beautiful and easier to clean. The front pushrod 92 is further provided with a precise locating sleeve 95 at the proximal end, to limit relative positions of the front pushrod 92 and the inner connection hose 91, so that the stent can be precisely mounted at a specific position of the implanter.

### Embodiment 8

As shown in FIG. 22 to FIG. 25,
an occluding stent 6 includes a distal flange occluding body 60, a proximal flange occluding body 61 and a connection portion 62; external surfaces of the distal flange occluding body 60, the proximal flange occluding body 61 and the connection portion 62 are all provided with a coating.

In addition, a first occluding coating 63 is provided between a first occluding portion end 65 on an external side of the distal flange occluding body 60 and an inner cavity of the connection portion 62; a second occluding coating 64 is provided between a second occluding end 66 on an external side of the proximal flange occluding body 61 and the inner cavity of the connection portion 62.

The distal flange occluding body 60 shapes like a bowl that is concave inward.

The first occluding coating 63 is provided between a diameter shrinkage area of the first occluding portion end 65 and the inner cavity of the connection portion 62.

The proximal flange occluding body 61 is umbrella-shaped or flare-shaped.

The second occluding coating 64 is provided between a diameter shrinkage area of the second occluding end 66 and the inner cavity of the connection portion 62.

Food or foreign objects are prevented from entering the trachea from the esophagus.

The coatings in this embodiment may be silicone coatings meeting medical standards or may be materials having a similar function.

In the longitudinal direction A of the occluding stent 6, a distalmost end of the distal flange occluding body 60, i.e. an end surface 65′ of a first occluding portion end 65 of the distal flange occluding body 60, is flat when viewed from the radial direction B of the occluding stent 6, and has no outward protrusion in the longitudinal direction A away from the connection portion 62. Similarly, a proximalmost end of the proximal flange occluding body 61, i.e., an end surface 66′ of a second occluding portion end 66 of the proximal flange occluding body 61, is flat when viewed from the radial direction B of the occluding stent 6, and has no outward protrusion in the longitudinal direction A away from the connection portion 62. The radial direction B can be parallel to the end surface 65′ of the first occluding portion end 65 of the distal flange occluding body 60 and the end surface 66′ of the second occluding portion end 66 of the proximal flange occluding body 61, in other words, the radial direction B can be the radial directions of the distal flange occluding body 60, the proximal flange occluding body 61 and the connection portion 62, and the radial direction B can be perpendicular to the longitudinal direction A.

The end surface 65′ of the first occluding portion end 65 of the distal flange occluding body 60 and the end surface 66′ of the second occluding portion end 66 of the proximal flange occluding body 61 are all in the shape having an opening in their middle portions, and an edge of the opening is at a certain distance from a center axis C in the longitudinal direction A of the occluding stent. It can be seen in the figure that the entire end surfaces 65′ and 66′ are not completely braided by metal wires and the middle portions of the end surfaces are empty and not braided. It can be seen from FIG. 23 that dotted line representing the longitudinal direction A of the occluding stent can also represent the center axis C in the longitudinal direction A of the occluding stent.

Thus, the proximal and distal ends of the occluding stent do not have end covers or tails protruding outwardly, thereby preventing damage to surrounding tissues.

### Embodiment 9

As shown in FIG. 26 to FIG. 28,
an occluding stent 6 includes a distal flange occluding body 60, a proximal flange occluding body 61 and a connection portion 62; external surfaces of the distal flange occluding body 60, the proximal flange occluding body 61 and the connection portion 62 are all provided with a coating.

In addition, a first occluding coating 63 is provided between a first occluding portion end 65 on an external side of the distal flange occluding body 60 and an inner cavity of the connection portion 62; a second occluding coating 64 is provided between a second occluding end 66 on an external side of the proximal flange occluding body 61 and the inner cavity of the connection portion 62.

The distal flange occluding body 60 shapes like a bowl that is concave inward.

The first occluding coating 63 is provided between a diameter shrinkage area of the first occluding portion end 65 and the inner cavity of the connection portion 62.

The proximal flange occluding body 61 shapes like a bowl that is concave inward.

The second occluding coating 64 is provided between a diameter shrinkage area of the second occluding end 66 and the inner cavity of the connection portion 62.

Food or foreign objects are prevented from entering the trachea from the esophagus.

In addition, in this embodiment, a plurality of tantalum markers, for example, 4 tantalum markers, may be uniformly distributed on a single side of the stent.

The coatings in this embodiment may be silicone coatings meeting medical standards or may be materials having a similar function.

In the longitudinal direction A of the occluding stent 6, a distalmost end of the distal flange occluding body 60, i.e. an end surface 65′ of a first occluding portion end 65 of the distal flange occluding body 60, is flat when viewed from the radial direction B of the occluding stent 6, and has no outward protrusion in the longitudinal direction A away from the connection portion 62. Similarly, a proximalmost end of the proximal flange occluding body 61, i.e., an end surface 66′ of a second occluding portion end 66 of the proximal flange occluding body 61, is flat when viewed from the radial direction B of the occluding stent 6, and has no outward protrusion in the longitudinal direction A away from the connection portion 62. The radial direction B can be parallel to the end surface 65′ of the first occluding portion end 65 of the distal flange occluding body 60 and the end surface 66′ of the second occluding portion end 66 of the proximal flange occluding body 61, in other words, the radial direction B can be the radial directions of the distal flange occluding body 60, the proximal flange occluding body 61 and the connection portion 62, and the radial direction B can be perpendicular to the longitudinal direction A.

The end surface 65′ of the first occluding portion end 65 of the distal flange occluding body 60 and the end surface 66′ of the second occluding portion end 66 of the proximal flange occluding body 61 are all in the shape having an opening in their middle portions, and an edge of the opening is at a certain distance from a center axis C in the longitudinal direction A of the occluding stent. It can be seen in the figure that the entire end surfaces 65′ and 66′ are not completely braided by metal wires and the middle portions of the end surfaces are empty and not braided. It can be seen from FIG. 27 that dotted line representing the longitudinal direction A of the occluding stent can also represent the center axis C in the longitudinal direction A of the occluding stent.

Thus, the proximal and distal ends of the occluding stent do not have end covers or tails protruding outwardly, thereby preventing damage to surrounding tissues.

### Embodiment 10

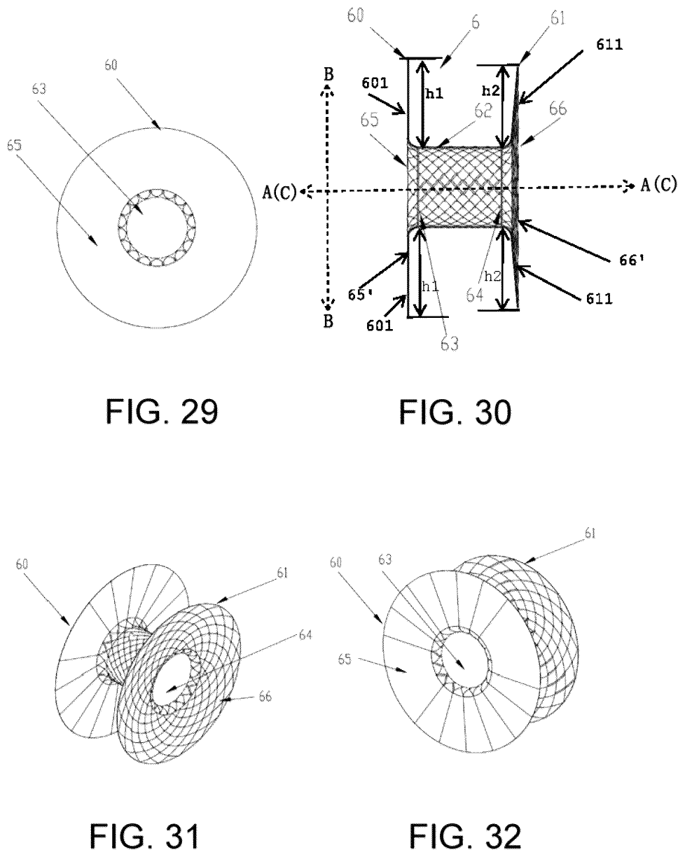

As shown in FIG. 29 to FIG. 32,
an occluding stent 6 includes a distal flange occluding body 60, a proximal flange occluding body 61 and a connection portion 62; external surfaces of the distal flange occluding body 60, the proximal flange occluding body 61 and the connection portion 62 are all provided with a coating.

In addition, a first occluding coating 63 is provided between a first occluding portion end 65 on an external side of the distal flange occluding body 60 and an inner cavity of the connection portion 62; a second occluding coating 64 is provided between a second occluding end 66 on an external side of the proximal flange occluding body 61 and the inner cavity of the connection portion 62.

The distal flange occluding body 60 is disc-shaped or umbrella-shaped.

The first occluding coating 63 is provided between a diameter shrinkage area of the first occluding portion end 65 and the inner cavity of the connection portion 62.

The proximal flange occluding body 61 is disc-shaped or umbrella-shaped.

The second occluding coating 64 is provided between a diameter shrinkage area of the second occluding end 66 and the inner cavity of the connection portion 62.

Food or foreign objects are prevented from entering the trachea from the esophagus.

The coatings in this embodiment may be silicone coatings meeting medical standards or may be materials having a similar function.

In the longitudinal direction A of the occluding stent 6, a distalmost end of the distal flange occluding body 60, i.e. an end surface 65′ of a first occluding portion end 65 of the distal flange occluding body 60, is flat when viewed from the radial direction B of the occluding stent 6, and has no outward protrusion in the longitudinal direction A away from the connection portion 62. Similarly, a proximalmost end of the proximal flange occluding body 61, i.e., an end surface 66′ of a second occluding portion end 66 of the proximal flange occluding body 61, is flat when viewed from the radial direction B of the occluding stent 6, and has no outward protrusion in the longitudinal direction A away from the connection portion 62. The radial direction B can be parallel to the end surface 65′ of the first occluding portion end 65 of the distal flange occluding body 60 and the end surface 66′ of the second occluding portion end 66 of the proximal flange occluding body 61, in other words, the radial direction B can be the radial directions of the distal flange occluding body 60, the proximal flange occluding body 61 and the connection portion 62, and the radial direction B can be perpendicular to the longitudinal direction A.

The end surface 65′ of the first occluding portion end 65 of the distal flange occluding body 60 and the end surface 66′ of the second occluding portion end 66 of the proximal flange occluding body 61 are all in the shape having an opening in their middle portions, and an edge of the opening is at a certain distance from a center axis C in the longitudinal direction A of the occluding stent. It can be seen in the figure that the entire end surfaces 65′ and 66′ are not completely braided by metal wires and the middle portions of the end surfaces are empty and not braided. It can be seen from FIG. 30 that dotted line representing the longitudinal direction A of the occluding stent can also represent the center axis C in the longitudinal direction A of the occluding stent.

Thus, the proximal and distal ends of the occluding stent do not have end covers or tails protruding outwardly, thereby preventing damage to surrounding tissues.

### Embodiment 11

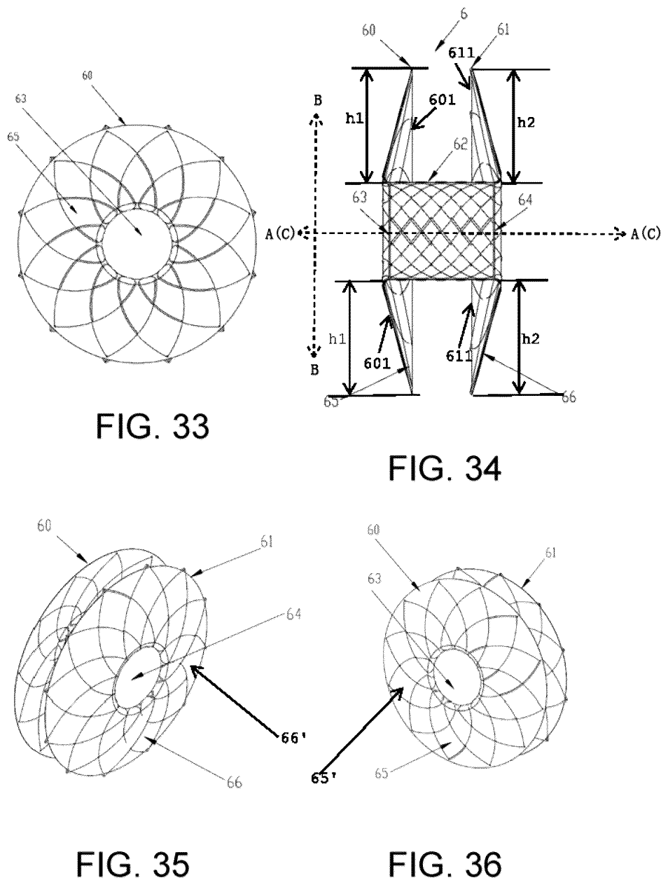

As shown in FIG. 33 to FIG. 36, an occluding stent 6 includes a distal flange occluding body 60, a proximal flange occluding body 61 and a connection portion 62; external surfaces of the distal flange occluding body 60, the proximal flange occluding body 61 and the connection portion 62 are all provided with a coating.

In addition, a first occluding coating 63 is provided between a first occluding portion end 65 on an external side of the distal flange occluding body 60 and an inner cavity of the connection portion 62; a second occluding coating 64 is provided between a second occluding end 66 on an external side of the proximal flange occluding body 61 and the inner cavity of the connection portion 62.

The distal flange occluding body 60 is umbrella-shaped, and provides coverage towards an internal side. That is, if the first occluding portion end 65 is compared to an umbrella surface, the umbrella surface faces outwards.

The first occluding coating 63 is provided between the first occluding portion end 65 and the inner cavity of the connection portion 62.

The proximal flange occluding body 61 is umbrella-shaped, and provides coverage towards an internal side. That is, if the first occluding portion end 65 is compared to an umbrella surface, the umbrella surface faces outwards.

The second occluding coating 64 is provided between the second occluding end 66 and the inner cavity of the connection portion 62.

Food or foreign objects are prevented from entering the trachea from the esophagus.

The coatings in this embodiment may be silicone coatings meeting medical standards or may be materials having a similar function.

In the longitudinal direction A of the occluding stent 6, a distalmost end of the distal flange occluding body 60, i.e. an end surface 65′ of a first occluding portion end 65 of the distal flange occluding body 60, is flat when viewed from the radial direction B of the occluding stent 6, and has no outward protrusion in the longitudinal direction A away from the connection portion 62. Similarly, a proximalmost end of the proximal flange occluding body 61, i.e., an end surface 66′ of a second occluding portion end 66 of the proximal flange occluding body 61, is flat when viewed from the radial direction B of the occluding stent 6, and has no outward protrusion in the longitudinal direction A away from the connection portion 62. The radial direction B can be parallel to the end surface 65′ of the first occluding portion end 65 of the distal flange occluding body 60 and the end surface 66′ of the second occluding portion end 66 of the proximal flange occluding body 61, in other words, the radial direction B can be the radial directions of the distal flange occluding body 60, the proximal flange occluding body 61 and the connection portion 62, and the radial direction B can be perpendicular to the longitudinal direction A.

The end surface 65′ of the first occluding portion end 65 of the distal flange occluding body 60 and the end surface 66′ of the second occluding portion end 66 of the proximal flange occluding body 61 are all in the shape having an opening in their middle portions, and an edge of the opening is at a certain distance from a center axis C in the longitudinal direction A of the occluding stent. It can be seen in the figure that the entire end surfaces 65′ and 66′ are not completely braided by metal wires and the middle portions of the end surfaces are empty and not braided. It can be seen from FIG. 34 that dotted line representing the longitudinal direction A of the occluding stent can also represent the center axis C in the longitudinal direction A of the occluding stent.

Thus, the proximal and distal ends of the occluding stent do not have end covers or tails protruding outwardly, thereby preventing damage to surrounding tissues.

### Embodiment 12

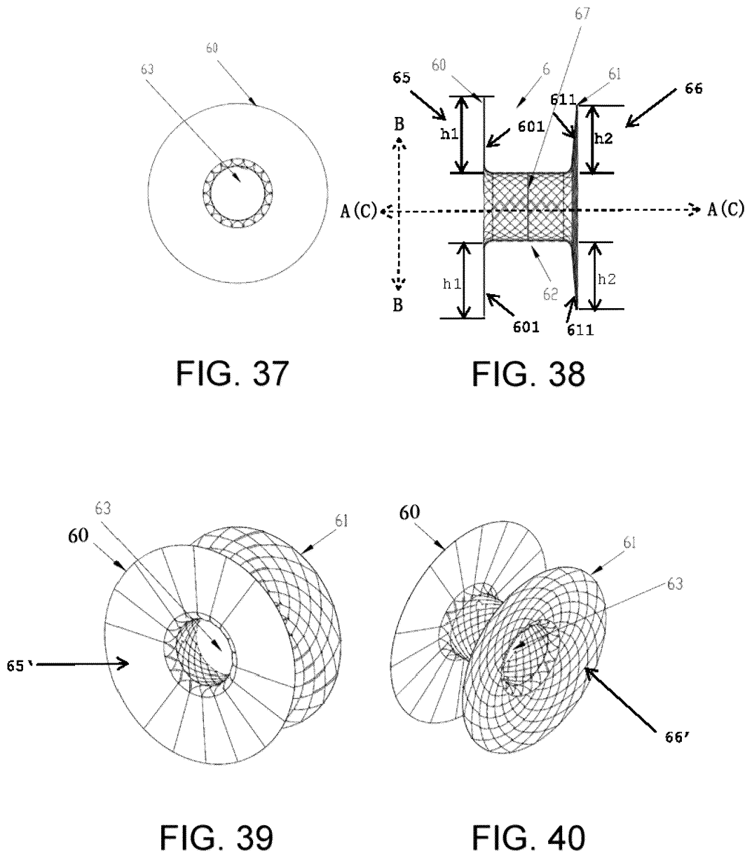

As shown in FIG. 37 to FIG. 40, an occluding stent 6 includes a distal flange occluding body 60, a proximal flange occluding body 61 and a connection portion 62; external surfaces of the distal flange occluding body 60, the proximal flange occluding body 61 and the connection portion 62 are all provided with a coating.

The distal flange occluding body 60 is umbrella-shaped or disc-shaped.

An inner cavity of the connection portion 62 is provided with a third occluding coating 67.

The proximal flange occluding body 61 is umbrella-shaped or disc-shaped.

The middle of a cavity channel of the connection portion 62 is plugged with silicone. Neither end of the connection portion 62 is plugged, or one of the two ends of the connection portion 62 is plugged. Food or foreign objects are prevented from entering the trachea from the esophagus.

The coatings in this embodiment may be silicone coatings meeting medical standards or may be materials having a similar function.

In the longitudinal direction A of the occluding stent 6, a distalmost end of the distal flange occluding body 60, i.e. an end surface 65′ of a first occluding portion end 65 of the distal flange occluding body 60, is flat when viewed from the radial direction B of the occluding stent 6, and has no outward protrusion in the longitudinal direction A away from the connection portion 62. Similarly, a proximalmost end of the proximal flange occluding body 61, i.e., an end surface 66′ of a second occluding portion end 66 of the proximal flange occluding body 61, is flat when viewed from the radial direction B of the occluding stent 6, and has no outward protrusion in the longitudinal direction A away from the connection portion 62. The radial direction B can be parallel to the end surface 65′ of the first occluding portion end 65 of the distal flange occluding body 60 and the end surface 66′ of the second occluding portion end 66 of the proximal flange occluding body 61, in other words, the radial direction B can be the radial directions of the distal flange occluding body 60, the proximal flange occluding body 61 and the connection portion 62, and the radial direction B can be perpendicular to the longitudinal direction A.

The end surface 65′ of the first occluding portion end 65 of the distal flange occluding body 60 and the end surface 66′ of the second occluding portion end 66 of the proximal flange occluding body 61 are all in the shape having an opening in their middle portions, and an edge of the opening is at a certain distance from a center axis C in the longitudinal direction A of the occluding stent. It can be seen in the figure that the entire end surfaces 65′ and 66′ are not completely braided by metal wires and the middle portions of the end surfaces are empty and not braided. It can be seen from FIG. 38 that dotted line representing the longitudinal direction A of the occluding stent can also represent the center axis C in the longitudinal direction A of the occluding stent.

Thus, the proximal and distal ends of the occluding stent do not have end covers or tails protruding outwardly, thereby preventing damage to surrounding tissues.

### Embodiment 13

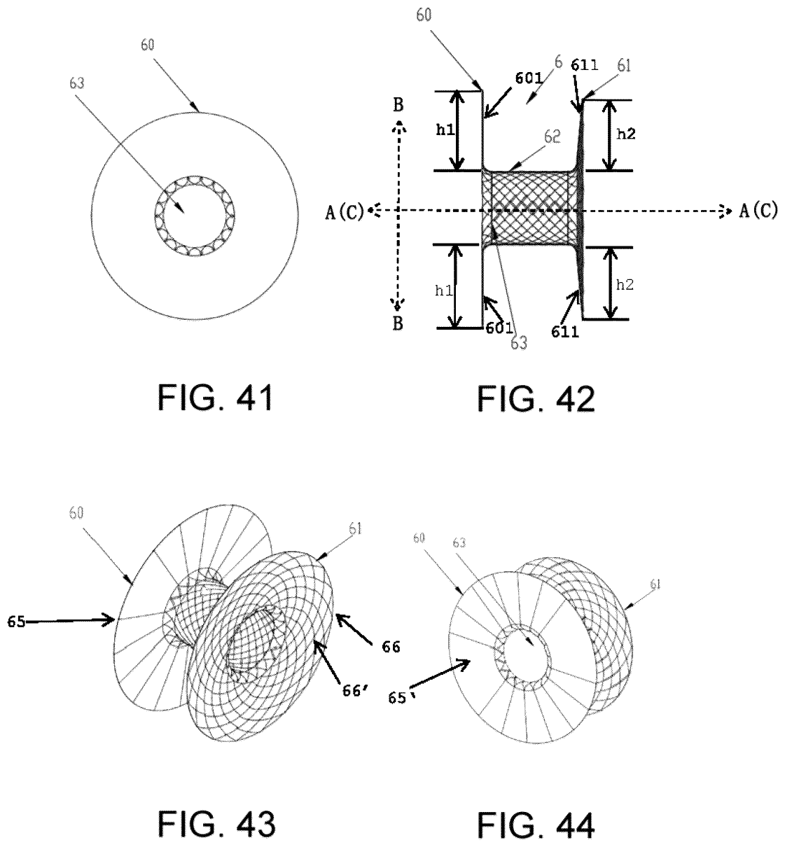

As shown in FIG. 41 to FIG. 44, an occluding stent 6 includes a distal flange occluding body 60, a proximal flange occluding body 61 and a connection portion 62; external surfaces of the distal flange occluding body 60, the proximal flange occluding body 61 and the connection portion 62 are all provided with a coating.

The distal flange occluding body 60 is disc-shaped or umbrella-shaped.

The proximal flange occluding body 61 is disc-shaped or umbrella-shaped.

A first occluding coating 63 is provided between a diameter shrinkage area of a first occluding portion end 65 and an inner cavity of the connection portion 62, or a second occluding coating 64 is provided between a diameter shrinkage area of a second occluding end 66 and the inner cavity of the connection portion 62. That is, the cavity channel at only one end of the connection portion 62 is plugged with silicone, while the other end is not plugged.

The proximal flange occluding body 61 is disc-shaped or umbrella-shaped. In the figures, the first occluding coating 63 being provided between the diameter shrinkage area of the first occluding portion end 65 and the inner cavity of the connection portion 62 is used as an example.

Food or foreign objects are prevented from entering the trachea from the esophagus.

The coatings in this embodiment may be silicone coatings meeting medical standards or may be materials having a similar function.

In the longitudinal direction A of the occluding stent 6, a distalmost end of the distal flange occluding body 60, i.e. an end surface 65′ of a first occluding portion end 65 of the distal flange occluding body 60, is flat when viewed from the radial direction B of the occluding stent 6, and has no outward protrusion in the longitudinal direction A away from the connection portion 62. Similarly, a proximalmost end of the proximal flange occluding body 61, i.e., an end surface 66′ of a second occluding portion end 66 of the proximal flange occluding body 61, is flat when viewed from the radial direction B of the occluding stent 6, and has no outward protrusion in the longitudinal direction A away from the connection portion 62. The radial direction B can be parallel to the end surface 65′ of the first occluding portion end 65 of the distal flange occluding body 60 and the end surface 66′ of the second occluding portion end 66 of the proximal flange occluding body 61, in other words, the radial direction B can be the radial directions of the distal flange occluding body 60, the proximal flange occluding body 61 and the connection portion 62, and the radial direction B can be perpendicular to the longitudinal direction A.

The end surface 65′ of the first occluding portion end 65 of the distal flange occluding body 60 and the end surface 66′ of the second occluding portion end 66 of the proximal flange occluding body 61 are all in the shape having an opening in their middle portions, and an edge of the opening is at a certain distance from a center axis C in the longitudinal direction A of the occluding stent. It can be seen in the figure that the entire end surfaces 65′ and 66′ are not completely braided by metal wires and the middle portions of the end surfaces are empty and not braided. It can be seen from FIG. 42 that dotted line representing the longitudinal direction A of the occluding stent can also represent the center axis C in the longitudinal direction A of the occluding stent.

Thus, the proximal and distal ends of the occluding stent do not have end covers or tails protruding outwardly, thereby preventing damage to surrounding tissues.

### Embodiment 14

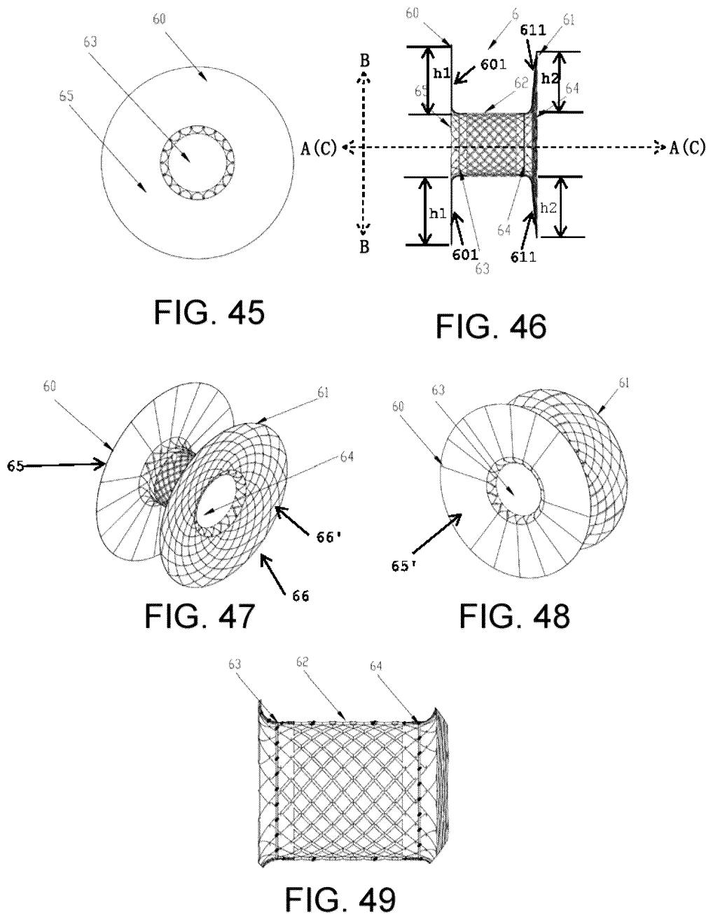

As shown in FIG. 45 to FIG. 49, an occluding stent 6 includes a distal flange occluding body 60, a proximal flange occluding body 61 and a connection portion 62; external surfaces of the distal flange occluding body 60 and the proximal flange occluding body 61 are both provided with a coating. An external surface of the connection portion 62 is partially provided with a coating. Specifically, a middle part of the external surface of the connection portion 62 is directly exposed, and both ends of the external surface are provided with the coating.

The distal flange occluding body 60 is disc-shaped or umbrella-shaped.

The proximal flange occluding body 61 is disc-shaped or umbrella-shaped.

A first occluding coating 63 is provided between a diameter shrinkage area of a first occluding portion end 65 and an inner cavity of the connection portion 62, and/or a second occluding coating 64 is provided between a diameter shrinkage area of a second occluding end 66 and the inner cavity of the connection portion 62.

Food or foreign objects are prevented from entering the trachea from the esophagus.

The coatings in this embodiment may be silicone coatings meeting medical standards or may be materials having a similar function.

In the foregoing embodiments, the placement position of the distal flange occluding body 60 is not limited to the esophageal side or the trachea side; the placement position of the proximal flange occluding body 61 is not limited to the esophageal side or the trachea side; the connection portion 62 is placed between the esophagus and the trachea, but it is not excluded that a small part of or more than half of the connection portion 62 is located on the esophageal side or the trachea side.

In the longitudinal direction A of the occluding stent 6, a distalmost end of the distal flange occluding body 60, i.e. an end surface 65′ of a first occluding portion end 65 of the distal flange occluding body 60, is flat when viewed from the radial direction B of the occluding stent 6, and has no outward protrusion in the longitudinal direction A away from the connection portion 62. Similarly, a proximalmost end of the proximal flange occluding body 61, i.e., an end surface 66′ of a second occluding portion end 66 of the proximal flange occluding body 61, is flat when viewed from the radial direction B of the occluding stent 6, and has no outward protrusion in the longitudinal direction A away from the connection portion 62. The radial direction B can be parallel to the end surface 65′ of the first occluding portion end 65 of the distal flange occluding body 60 and the end surface 66′ of the second occluding portion end 66 of the proximal flange occluding body 61, in other words, the radial direction B can be the radial directions of the distal flange occluding body 60, the proximal flange occluding body 61 and the connection portion 62, and the radial direction B can be perpendicular to the longitudinal direction A.

The end surface 65′ of the first occluding portion end 65 of the distal flange occluding body 60 and the end surface 66′ of the second occluding portion end 66 of the proximal flange occluding body 61 are all in the shape having an opening in their middle portions, and an edge of the opening is at a certain distance from a center axis C in the longitudinal direction A of the occluding stent. It can be seen in the figure that the entire end surfaces 65′ and 66′ are not completely braided by metal wires and the middle portions of the end surfaces are empty and not braided. It can be seen from FIG. 46 that dotted line representing the longitudinal direction A of the occluding stent can also represent the center axis C in the longitudinal direction A of the occluding stent.

Thus, the proximal and distal ends of the occluding stent do not have end covers or tails protruding outwardly, thereby preventing damage to surrounding tissues.

### Embodiment 15

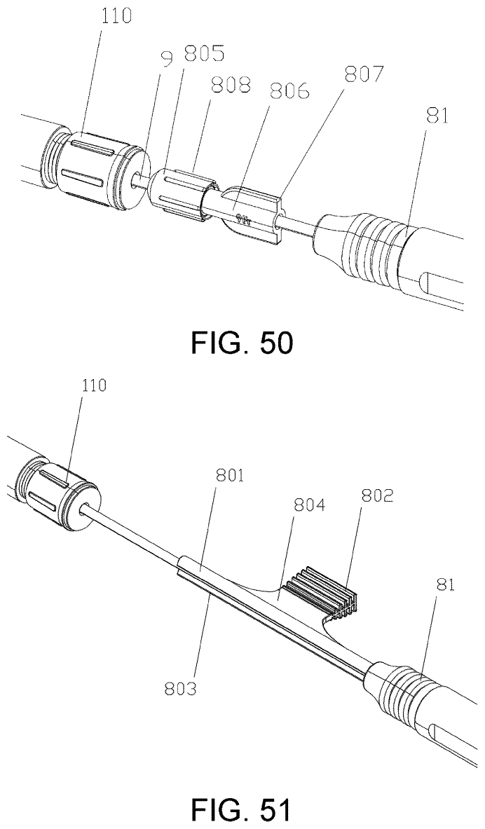

FIG. 50 is a schematic structural diagram of a range-adjustable mark in Embodiment 16 of the present application.

A range-adjustable mark is provided on a rear pushrod 9 between a rear pushrod locking knob 110 and a rear-handle front cover 81 of an implanter. The range-adjustable mark includes a positioning nut 805 at a distal end and a range adjuster 806 that is located at a proximal end and connected to the positioning nut 805 through threads. The positioning nut 805 is provided with an anti-slip rib 808, and the range adjuster 806 is provided with an alary protrusion 807. The positioning nut 805 and the range adjuster 806 are sleeved over the rear pushrod 9. The range adjuster 806 is screwed in or out, to adjust a distance between the rear pushrod locking knob 110 and the rear-handle front cover 81.

The range-adjustable mark is used for instructing the distal occluding portion body of the occluding stent to open. In the process of pushing the rear handle to the distal end, when the distance between the rear pushrod locking knob 110 and the rear-handle front cover 81 is completely occupied by the range-adjustable mark, it indicates that the rear handle is pushed to a predetermined position, that is, the distal occluding portion body of the occluding stent has been opened. Then, the rear handle is not pushed any more, and the implanter is pulled out towards the proximal end, so that the proximal occluding portion body of the occluding stent is open, to complete stent implanting and occlusion between the esophagus and the trachea.

The range-adjustable mark is applicable to an implanter through which a guide wire is passed or no guide wire is passed.

### Embodiment 16

FIG. 51 is a schematic structural diagram of a removable mark in Embodiment 17 of the present application.

A removable mark is provided on a rear pushrod 9 between a rear pushrod locking knob 110 and a rear-handle front cover 81 of an implanter. The removable mark is sleeved over the rear pushrod 9 and includes a removable sleeve 80 land an anti-slip handle 802. The removable sleeve 801 and the anti-slip handle 802 are connected through a connector 804. A notch 803 for allowing the rear pushrod 9 to enter is provided on a side of the removable sleeve 801. The removable mark may be sleeved over the rear pushrod 9 through the notch 803. The connection is implemented relying on an elastic force of the removable sleeve 801, so that the distance between the rear pushrod locking knob 110 and the rear-handle front cover 81 is locked. The removable mark is used for instructing the distal occluding portion body of the occluding stent to open. In the process of pushing the rear handle to the distal end, when the distance between the rear pushrod locking knob 110 and the rear-handle front cover 81 is completely occupied by the removable mark, it indicates that the rear handle is pushed to a predetermined position, that is, the distal occluding portion body of the occluding stent has been opened. Then, the rear handle is not pushed any more, and the implanter is pulled out towards the proximal end, so that the proximal occluding portion body of the occluding stent is open, to complete stent implanting and occlusion between the esophagus and the trachea.

The removable mark is applicable to an implanter through which a guide wire is passed or no guide wire is passed.

### Embodiment 17

The implanter for an occluding stent is also provided with an inner hole for a guide wire to pass through one side of the occluding stent.

### Embodiment 18

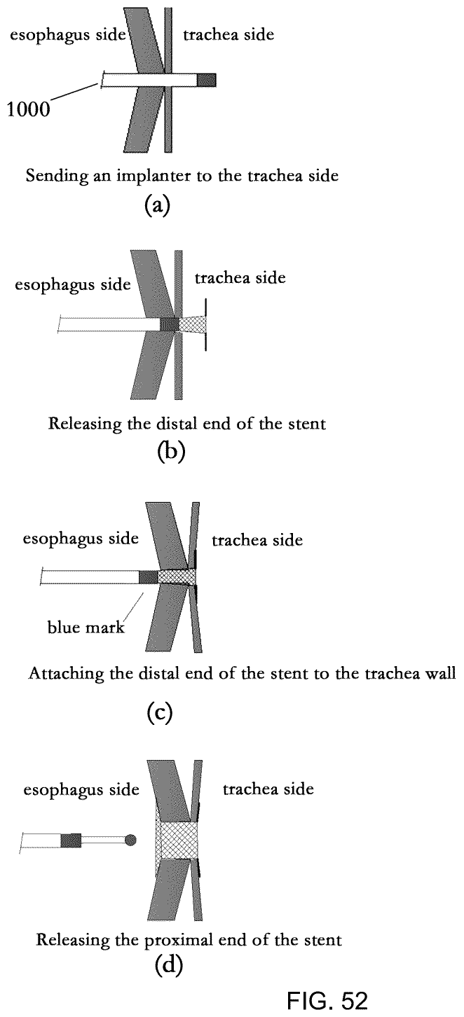

As shown in FIG. 52, a method for implanting an implanter for a fistula having a size of less than 9 mm includes the following steps:

S1 feeding a tracheoscope through the nose, feeding a gastroscope or colonoscope having a clamp channel of great than or equal to 3.2 mm through the mouth, and locating the position of the fistula by the combination of two scopes;

S2 after the position of the fistula is confirmed, feeding the implanter 1000 described in the above embodiments through the clamp channel of the gastroscope or colonoscope to cause the distal end of the implanter 1000 to pass through the fistula from an esophageal side to a tracheal side under the supervision of the gastroscope or colonoscope (as shown in (a) of FIG. 52), and confirming under the supervision of the tracheoscope that the distal end of the implanter 1000 has entered a trachea and has not scratched an inner wall of the trachea, where an occluding stent is loaded into the flexible tube 94 at the distal end of the implanter 1000, and the occluding stent abuts against the front pusher 93, and the connection portion of the occluding stent has a diameter D1 of 2 mm to 9 mm;

S3 unlocking the front handle 115 of the implanter 1000 from the rear actuator locking knob 110 of the implanter 1000 to allow the front handle 115 to be slidable relative to the rear pushrod 9, and pulling the front handle 115 of the implanter 1000 backward to a first target 119 so as to release the distal flange occluding body of the occluding stent under the supervision of the tracheoscope, and confirming that the distal flange occluding body of the occluding stent is unfolded (as shown in (b) of FIG. 52);

S4 slowly moving the implanter 1000 backward until a color mark (e.g., a blue mark) on the distal end of the flexible tube 94 of the implanter 1000 is observed under the gastroscope or colonoscope to have returned to the esophageal side (as shown in (c) of FIG. 52), pulling the front handle 115 of the implanter 1000 backward towards the proximal end to release the proximal flange occluding body of the occluding stent, and confirming under gastroscope or colonoscope that the proximal flange occluding body has been released and unfolded, and the occluding stent does not fall off (as shown in (d) of FIG. 52);
S5 withdrawing the implanter 1000 out of the body;
S6 injecting a methylene blue mucosal stain through the clamp channel of the gastroscope or colonoscope, and then confirming whether the methylene blue mucosal stain is leaked to the tracheal side through the tracheoscope, and withdrawing all endoscopes if there is no leakage.

### Embodiment 19

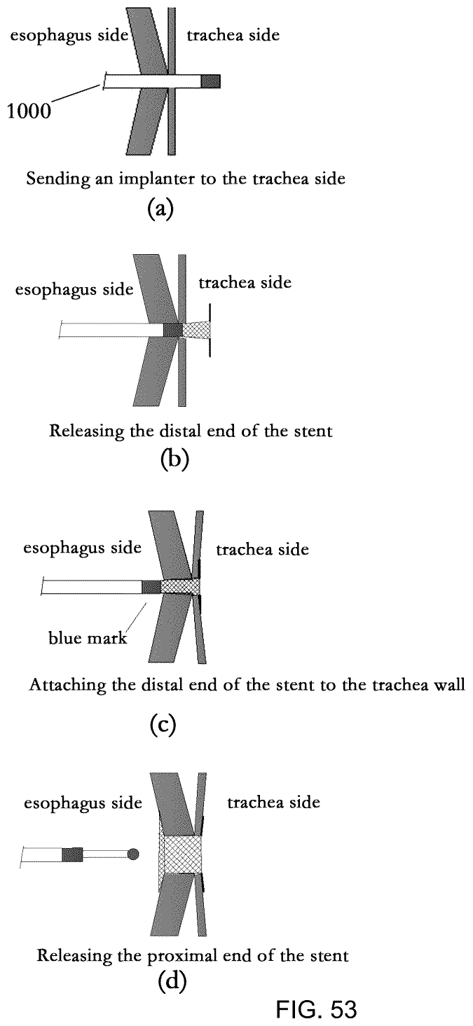

As shown in FIG. 53, a method for implanting an implanter for a fistula having a size of greater than 9 mm includes the following steps:
S1 feeding a tracheoscope through the nose, feeding a ultrafine gastroscope and an over tube through the mouth (the ultrafine gastroscope is placed in the over tube), and locating the position of the fistula by the combination of two scopes;
S2 after the position of the fistula is confirmed, feeding the distal end of the ultrafine gastroscope to pass through the fistula from an esophageal side to a tracheal side, and under the supervision of the tracheoscope, feeding the distal end of the over tube to the tracheal side along the ultrafine gastroscope, and withdrawing the ultrafine gastroscope from the over tube to the outside of the body;
S3 under the supervision of the tracheoscope, feeding the distal end of the implanter 1000 described in the above embodiments through an inner lumen of the over tube to the tracheal side (as shown in (a) of FIG. 53), confirming the distal end of the implanter 1000 to have entered the tracheal side, leaving the implanter 1000 in place and moving the over tube backward into the esophagus, where an occluding stent is loaded into the flexible tube 94 at the distal end of the implanter 1000, and the occluding stent abuts against the front pusher 93, and the connection portion of the occluding stent has a diameter D1 of 9 mm to 24 mm;
S4 feeding the ultrafine gastroscope along the outside of the over tube to the fistula at the esophageal side to observe the placement of the implanter at the esophageal side, unlocking the front handle 115 of the implanter 1000 from the rear actuator locking knob 110 of the implanter 1000 to allow the front handle 115 to be slidable relative to the rear pushrod 9, and pulling the front handle 115 of the implanter 1000 backward to a first target 119 so as to release the distal flange occluding body of the occluding stent under the supervision of the tracheoscope, and confirming that the distal flange occluding body of the occluding stent is unfolded (as shown in (b) of FIG. 52);
S5 moving the implanter 1000 backward to the flexible tube 94 of the implanter 1000 so as to fully expose the distal portion of the implant, slowly moving the implanter 1000 backward until a color mark (e.g., a blue mark) on the distal end of the flexible tube 94 of the implanter 1000 is observed under the ultrafine gastroscope to have returned to the esophageal side (as shown in (c) of FIG. 53), pulling the front handle 115 of the implanter 1000 backward towards the proximal end to release the proximal flange occluding body of the occluding stent, and after the releasing of the proximal flange occluding body is completed, confirming under the ultrafine gastroscope that the proximal flange occluding body has been unfolded, and the occluding stent does not fall off (as shown in (d) of FIG. 53);
S6 withdrawing the implanter 1000 and the over tube out of the body;
S7 injecting a methylene blue mucosal stain through the clamp channel of the gastroscope or colonoscope, and then confirming whether the methylene blue mucosal stain is leaked to the tracheal side through the tracheoscope, and withdrawing all endoscopes if there is no leakage.

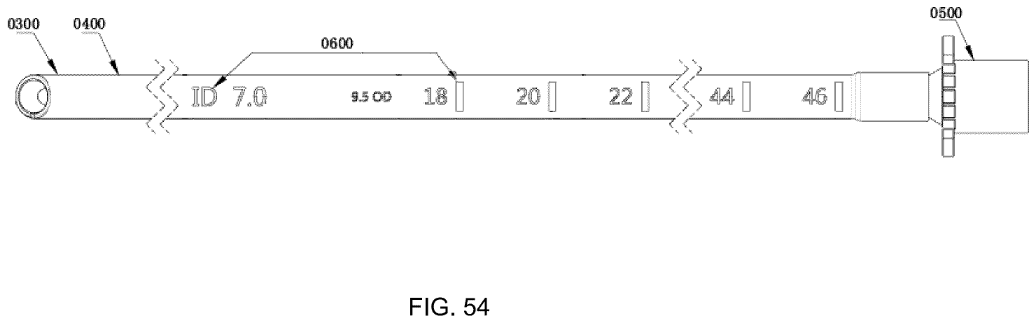

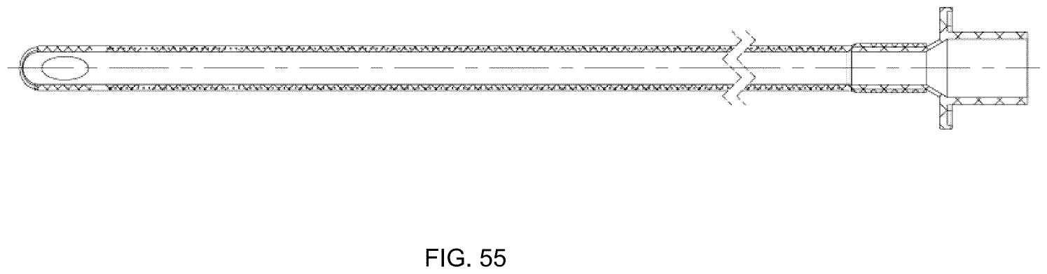

The structure of the over tube is shown in FIG. 54 and FIG. 55. Referring to the cross-sectional view of the over tube of FIG. 55, it can be seen that the two ends of the tubular part of the over tube are made of a medical high transparent polyvinyl chloride PVC material, as shown with the reference number of 0300; the middle portion of the tubular part of the over tube includes a stainless steel spring as shown with the reference number of 0400 in addition to the highly transparent polyvinyl chloride PVC; and a proximal end of the tubular part of the over tube is connected with a ABS (acrylonitrile-butadiene-styrene copolymer) handle through glue. There are also some black printed marks on the surface of the tubular part of the over tube, and the printed numbers are 18, 20, 22, 24, 26, 28, 30, 3234, 36, 38, 40, 42, 44, 46. The total length of the over tube is 502 mm±2 mm. The tip of the distal end of the over tube is formed by being cutting into a bevel. The inner diameter of the tubular part of the over tube is 7 mm±0.3 mm, and the outer diameter of the tubular part of the over tube is 9.5 mm±0.25 mm.

### Embodiment 20

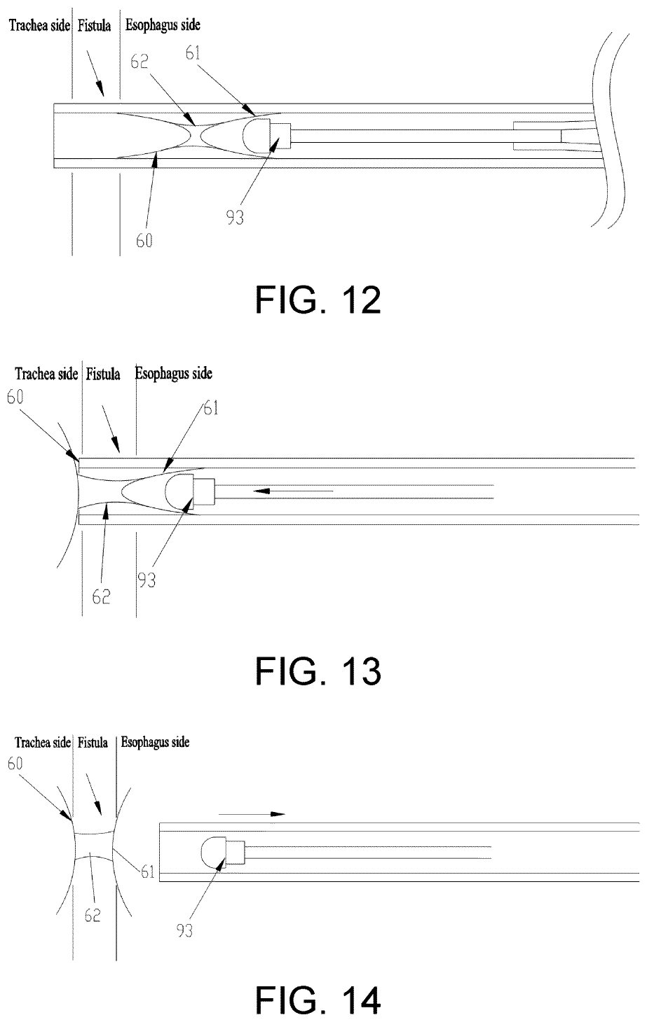

A method for implanting an implanter including an occluding stent for a fistula, including the following steps:
S1 locating the position of the fistula under a gastroscope;
S2 under the supervision of gastroscope, feeding the flexible tube 94, loading the occluding stent, of the implanter 1000 described in the above embodiments to pass through the fistula from the esophageal side to the tracheal side, as shown in FIG. 12;
S3 unlocking the front handle 115 of the implanter 1000 from the rear actuator locking knob 110 of the implanter 1000 to allow the front handle 115 to be slidable relative to the rear pushrod 9, pushing the back handle 8 to the distal end to cause the front pusher 93 to push the distal flange occluding body 60 of the occluding stent out of the flexible tube 94, so as to release the distal flange occluding body 60 at the tracheal side, as shown in FIG. 13;
S4 locking the front handle 115 to the rear actuator locking knob 110 so that the rear pushrod 9 and the front handle 115 are fixed, moving the implanter 1000 backward towards the distal end to cause the distal flange occluding body 60 to be blocked by an endotracheal wall, and then pulling the proximal flange occluding body 61 of the occluding stent out and releasing on the esophageal side, as shown in FIG. 14;
S5 confirming that the occluding stent is in the ideal position, then withdrawing the implanter 1000 out of the body, observing with the gastroscope again, and withdrawing the gastroscope if there is no abnormal situation.

### Embodiment 20

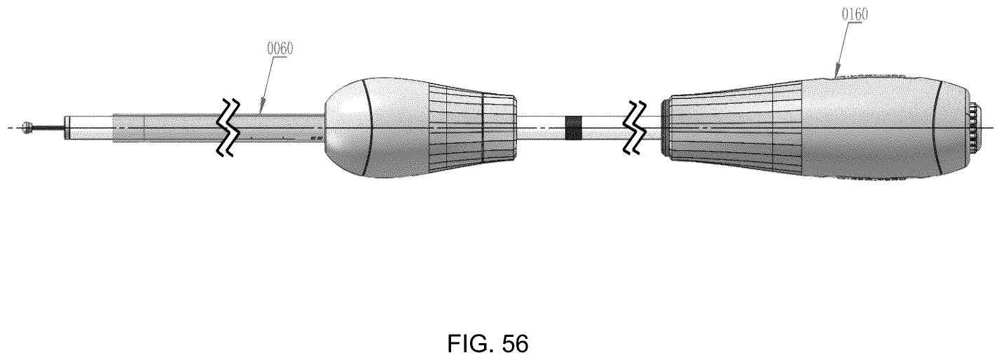

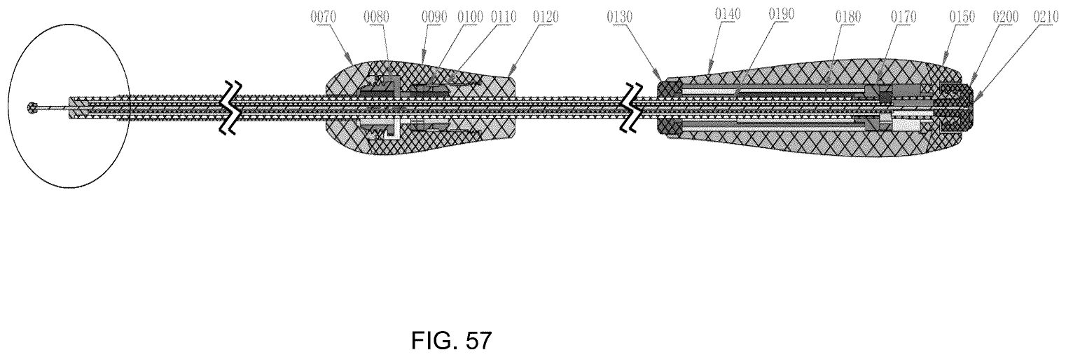

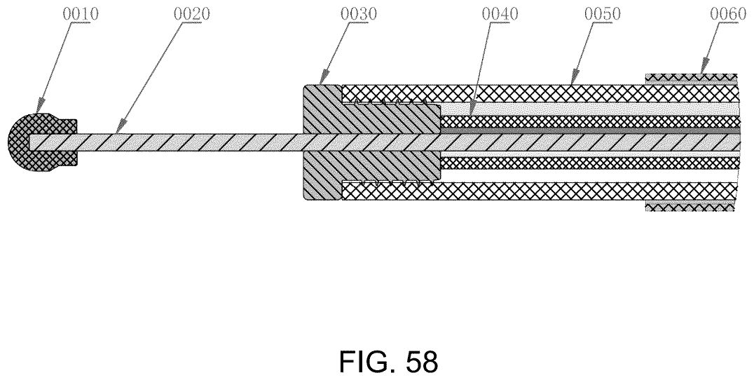

FIG. 56 is a schematic diagram of an overall assembly structure of an implanter according to one embodiment of the present application. FIG. 57 is a schematic diagram of a cross-sectional structure of the implanter of FIG. 56 of the present application. FIG. 58 shows a partially enlarged view of a front portion of an implanter of the present application.

An implanter for an occluding stent, including an outer tube 0060, a middle tube 0050, a front handle 0090 and a rear handle 0140. The proximal end of the middle tube 0050 is fixedly connected with the rear handle 0140, the middle tube 0050 passes through an interior of a middle tube locking knob 0120, then pass through an interior of the front handle 0090. The distal end of the middle tube 0050 is connected to a developing ring 0030, an inner tube 0040 is disposed in the middle tube 0050, the proximal end of the inner tube 0040 is also fixedly connected with rear handle 0140, the distal end of the inner tube 0040 is fixed to the developing ring 0030. The inner tube 0040 can be omitted, an inner core 0020 is disposed in the inner tube 0040, and the proximal end of the inner core 0020 is also fixedly connected with rear handle 0140, the distal end of the inner core 0020 passes through the developing ring 0030 and extends outward for a certain distance, and a front pusher 0010 is disposed on the distal end of the inner core 0020.

The proximal end of the outer tube 0060 is fixedly connected with the front handle 0090, the distal end of the outer tube 0060 extends out of the front handle 0090 at a certain distance, the outer tube 0060 is sleeved outside the middle tube 0050, and the front handle 0090 can be locked with or unlocked from the middle tube locking knob 0120, so that the front handle 0090 and the outer tube 0060 can be fixable or slidable relative to the rear handle 0140 and the middle tube 0050.

The occluding stent can be loaded into the outer tube 0060 and abuts against the front pusher 0010. The distal end of the front handle 0090 is connected to the front-handle front cover 0070 through a connecting sleeve 0080, the proximal end of the front handle 0090 is in threaded connection with the middle pipe locking knob 0120, so that the front handle 0090 can be fixedly connected to the middle tube 0050. The distal end of the middle pipe locking knob 0120 is provided with a locking ring 0110, the locking ring 0110 is in contact with the inner wall 0111 inside the front handle 0090, the distal end of the locking ring 0110 is provided with a sealing ring 0100, and when the middle pipe locking knob 0120 is tightened, the locking ring 0110 holds the middle pipe 0060 tightly.

The inner core 0020 is a metal rod, and the front pusher 0010, the rear handle 0140 and the front handle 0090 can be made of plastic through molds. The outer tube 0060, the middle tube 0050 and the inner tube 0040 are flexible hoses.

The distal end of the rear handle 0140 is connected to a rear-handle front cover 0130, the proximal end of the rear handle 0140 is connected to a Luer taper 0150, the proximal end of the Luer taper 0150 is connected to the rear-handle rear cover 0200, and the rear-handle rear cover 0200 is provided with a tail cover cap 0210. The middle tube 0050 and the inner tube 0040 are fixedly connected with the rear handle 0140 through a fixing ring 0170, and the inner core 0020 passes through the fixing ring 0170 and is connected to the rear-handle rear cover 0200.

The Luer taper 0150 can be connected to a syringe, etc., and can be used to flush or pump the middle tube 0050 and the inner tube 0040.

Basic principles, main features and advantages of the present application are shown and described above. A person skilled in the art should understand that the present application is not limited to the foregoing embodiments. The foregoing embodiments and the embodiments described in the specification are merely exemplary embodiments of the present application, which are not intended to limit the present application. The present application may have various changes and improvements without departing from the spirit and scope of the present application, and all these changes and improvements fall within the protection scope of the present application. The protection scope of the present application is subject to the appended claims and equivalents thereof.

What is claimed is:

1. An implanter comprising an outer tube, a middle tube, a front handle, and a rear handle, wherein a proximal end of the outer tube is fixedly connected to the front handle, the middle tube is located in the outer tube, a proximal end of the middle tube is fixedly connected to the rear handle, and the front handle and the outer tube are slidable relative to the rear handle and the middle tube;
a pushrod is fixedly mounted in the middle tube, and the pushrod extends beyond a distal end of the middle tube; an occluding stent is loaded into a distal end of the outer tube, and the occluding stent abuts against a distal end of the pushrod;
the implanter is configured such that the front handle is capable of being pulled backward towards a proximal end or the rear handle is capable of being pushed forward towards a distal end so as to expose and unfold the occluding stent.

2. An implanter comprising a rear handle, a rear pushrod, and a front handle, wherein a flexible tube is fixedly connected to a distal end of the front handle, a proximal end of the rear pushrod is fixedly connected to the rear handle, a distal end of the rear pushrod passes through an interior of a rear pushrod locking knob and then passes through an interior of the front handle to be connected to a front pushrod, a distal end of the front pushrod is connected to a front pusher, and the front pushrod is located in the flexible tube; the front handle is lockable to or unlockable from the rear pushrod locking knob to cause the front handle to be fixed with the rear pushrod or to cause the front handle to be slidable relative to the rear pushrod, wherein an occluding stent is loaded into a distal end of the flexible tube and the occluding stent abuts against the front pusher,
the implanter is configured such that the front handle is capable of being pulled backward towards a proximal end or the rear handle is capable of being pushed forward towards a distal end so as to expose and unfold the occluding stent.

3. The implanter according to claim 2, wherein a proximal end of the rear handle is provided with a rear-handle rear cover, a distal end of the rear handle is connected with a rear-handle front cover, and the rear pushrod is fixedly connected to the rear handle through a rear pushrod holder, and the distal end of the rear pushrod is connected to the front pushrod through an inner connection hose;
the front handle is provided with a Luer taper tube in communication with an inner cavity of the front handle, the distal end of the front handle is connected to a front-handle front cover through a connecting sleeve, and a distal end of the front-handle front cover is provided with a flexible sleeve hose connecting tube; the flexible sleeve hose connecting tube is connected to the flexible tube;
a distal end of the rear pushrod locking knob is provided with a tapered rubber plug, and the tapered rubber plug is in contact with a tapered inner wall inside the front handle, the tapered rubber plug is provided with a through hole for the rear pushrod to pass through, the tapered rubber plug has a tapered surface in contact with the tapered inner wall, and the tapered rubber plug is provided with a cross recess, such that when a proximal end of the front handle is in threaded connection with the rear pushrod locking knob, the tapered rubber plug holds the rear pushrod tightly.

4. The implanter according to claim 2, wherein a range-adjustable mark is provided on the rear pushrod between the rear pushrod locking knob and a rear-handle front cover of the implanter, the range-adjustable mark comprises a positioning nut located at the distal end and a range adjuster that is located at the proximal end and is in thread connection with the positioning nut, the positioning nut is provided with an anti-slip rib, the range adjuster is provided with an alary protrusion, the positioning nut and the range adjuster are sleeved over the rear pushrod, and the range adjuster is screwed in or out to adjust a distance between the rear pushrod locking knob and the rear-handle front cover.

5. The implanter according to claim 2, wherein a removable mark is provided on the rear pushrod between the rear pushrod locking knob and a rear-handle front cover of the implanter, the removable mark is sleeved over the rear pushrod and comprises a removable sleeve and an anti-slip handle, the removable sleeve and the anti-slip handle are connected through a connector, a notch for allowing the rear pushrod to enter is provided on a side of the removable sleeve, the removable mark is sleeved over the rear pushrod through the notch, and a connection is implemented replying on an elastic force of the removable sleeve, so that a distance between the rear pushrod locking knob and the rear-handle front cover is locked.

6. An occluding stent, comprising a distal flange occluding body, a proximal flange occluding body, and a connection portion, the distal flange occluding body and the proximal flange occluding body are respectively connected to two ends of the connection portion,
wherein in a longitudinal direction A of the occluding stent, an end surface of a distalmost end of the distal flange occluding body and an end surface of a proximalmost end of the proximal flange occluding body are both configured to have an opening at a middle portion of each of the end surfaces such that an edge of the opening is at a distance from a center axis C in the longitudinal direction A of the occluding stent.

7. The occluding stent according to claim 6, wherein the distal flange occluding body is umbrella-shaped, mushroom cap-shaped, cup-shaped, disc-shaped, bowl-shaped, hemi-spherical or spherical, the proximal flange occluding body is umbrella-shaped, mushroom cap-shaped, flare-shaped, cup-shaped or spherical, and a sealed occlusion state is formed between the distal flange occluding body and the proximal flange occluding body by using a coating.

8. The occluding stent according to claim 6, wherein a surface of the connection portion is wholly or partially provided with a coating; a surface of the distal flange occluding body is wholly or partially provided with a coating; and a surface of the proximal flange occluding body is wholly or partially provided with a coating.

9. The occluding stent according to claim 6, wherein a first occluding coating is provided between the distal flange occluding body and an inner cavity of the connection portion, and/or a second occluding coating is provided between the proximal flange occluding body and the inner cavity of the connection portion, and/or a third occluding coating is provided in the inner cavity of the connection portion; the first occluding coating, the second occluding coating, and the third occluding coating are disposed to be extended along a vertical direction of the occluding stent perpendicular to the longitudinal direction A to prevent food or foreign objects from passing through the inner cavity of the connection portion.

10. The occluding stent according to claim 6, wherein a portion of the distal flange occluding body extending beyond the connection portion in a radial direction B perpendicular to the longitudinal direction A is formed as a first shoulder, and a portion of the proximal flange occluding body extending beyond the connection portion in the radial direction B is formed as a second shoulder, such that the occluding stent is configured such that the connection portion is located in a fistula, and the first shoulder of the distal flange occluding body and the second shoulder of the proximal flange occluding body are seated on two side walls of the fistula respectively to seal the fistula,
wherein the first shoulder has a height h1 of 2-10 mm in the radial direction B and the second shoulder has a height h2 of 2-10 mm in the radial direction B.

11. The occluding stent according to claim 6, wherein the connection portion of the occluding stent is tubular, the connection portion has a diameter D1 in a range of greater than 0 and less than or equal to 10 mm, or the connection portion has a diameter D1 in a range of greater than or equal to 9 and less than or equal to 24 mm.

12. The occluding stent according to claim 6, wherein the distance from the edge of the opening to the central axis C in the longitudinal direction A of the occluding stent is greater than 0 and less than or equal to 23 mm.

13. A method for implanting the implanter according to claim 2, comprising steps of:
locating a position of a fistula, wherein the fistula is located between a first body channel and a second body channel;
feeding a distal end of the implanter to pass through the fistula from a first side of the fistula in the first body channel to a second side of the fistula in the second body channel, wherein an occluding stent is loaded into the flexible tube at the distal end of the implanter, the occluding stent comprises a distal flange occluding body, a proximal flange occluding body, and a connection portion, the distal flange occluding body and the proximal flange occluding body are respectively connected to two ends of the connection portion, wherein in a longitudinal direction A of the occluding stent, an end surface of a distalmost end of the distal flange occluding body and an end surface of a proximalmost end of the proximal flange occluding body are both configured to have an opening at a middle portion of each end surface such that an edge of the opening is at a distance from a center axis C in the longitudinal direction A of the occluding stent;
releasing the distal flange occluding body of the occluding stent from the distal end of the flexible tube to unfold the distal flange occluding body of the occluding stent at the second side of the fistula in the second body channel;
moving the implanter backward towards the proximal end to make the distal end of the flexible tube return to the first side of the fistula in the first body channel, and releasing the proximal flange occluding body of the occluding stent from the distal end of the flexible tube to unfold the proximal flange occluding body of the occluding stent at the first side of the fistula in the first body channel; and
withdrawing the implanter to an outside of a body.

14. The method according to claim 13, wherein the first body channel is an alimentary canal and the second body channel is an airway.

15. The method according to claim 13, wherein after the withdrawing the implanter to the outside of the body, the method further comprises injecting a methylene blue mucosal stain to the first side of the fistula, confirming whether the methylene blue mucosal stain is leaked to the second side of the fistula so as to confirm whether the occluding stent completely closes the fistula.

16. The method according to claim 13, wherein:
when the fistula has a size of less than 9 mm, the method comprises steps of:
feeding a first endoscope having a clamp channel of great than or equal to 3.2 mm into the first body channel, and feeding a second endoscope into the second body channel, and locating the position of the fistula in combination with the first endoscope and the second endoscope, the fistula is located between the first body channel and the second body channel;
feeding the implanter through the clamp channel of the first endoscope to allow the distal end of the implanter to pass through the fistula from the first side of the fistula in the first body channel to the second side of the fistula in the second body channel under the supervision of the first endoscope, and confirming that the distal end of the implanter has entered the second side of the fistula under the supervision of the second endoscope, wherein the occluding stent is loaded in the flexible tube at the distal end of the implanter and abuts against the front pusher, and the connection portion of the occluding stent has a diameter D1 of 2 mm to 9 mm;
unlocking the front handle of the implanter from the rear pushrod locking knob of the implanter to allow the front handle to be slidable relative to the rear pushrod, and pulling the front handle of the implanter backward towards the proximal end under the supervision of the second endoscope to allow the flexible tube to be moved backward together, so that the distal flange occluding body of the occluding stent is exposed and released from the flexible tube and is unfolded at the second side of the fistula in the second body channel; or pushing the rear handle forward towards the distal end to cause the front pusher to push the distal flange occluding body of the occluding stent out of the flexible tube, so that the distal flange occluding body of the occluding stent is released and unfolded at the second side of the fistula in the second body channel;
slowly moving the implanter backward towards the proximal end until the distal end of the flexible tube of the implanter is observed under the first endoscope to have returned to the first side of the fistula in the first body channel, pulling the front handle of the implanter backward towards the proximal end to cause the flexible tube to be moved backward together, so that the proximal flange occluding body of the occluding stent is exposed and released from the flexible tube and is unfolded at the first side of the fistula in the first body channel; or locking the front handle to the rear pushrod locking knob to cause the rear pushrod to be fixed to the front handle, and moving the implanter backward towards the proximal end, so that the distal flange occluding body is blocked by an inner wall of the second human second channel where the second side of the fistula is located and thus the proximal flange occluding body of the occluding stent is pulled out of the flexible tube, released and unfolded at the first side of the fistula in the first body channel; and
withdrawing the implanter to the outside of the body.

17. The method according to claim 16, wherein the first endoscope is a gastroscope or a colonoscope, and the second endoscope is a tracheoscope.

18. The method according to claim 13, wherein:
when the fistula has a size of greater than 9 mm, the method comprises steps of:
feeding a first endoscope and an over tube into the first body channel, wherein the first endoscope is placed inside the over tube, and feeding a second endoscope into a second body channel, and locating the position of the fistula in combination with the first endoscope and the second endoscope, wherein the fistula is located between the first body channel and the second body channel;
feeding a distal end of the first endoscope to pass through the fistula from the first side of the fistula in the first body channel to the second side of the fistula in the second body channel, feeding a distal end of the over tube along the first endoscope to the second side of the fistula in the second body channel under the supervision of the second endoscope, and then withdrawing the first endo scope out of the over tube to the outside of the body;
feeding a distal end of the implanter through an inner lumen of the over tube to the second side of the fistula in the second body channel under the supervision of the second endoscope, leaving the distal end of the implanter at the second side of the fistula in the second body channel, and moving the over tube backward to the first side of the fistula in the first body channel, wherein the occluding stent is loaded in the flexible tube at the distal end of the implanter and abuts against the front pusher, and the connection portion of the occluding stent has a diameter D1 of 9 mm to 24 mm;
feeding the first endoscope along an outer side of the over tube to the first side of the fistula in the first body channel to observe the placement of the implanter in the first body channel; unlocking the front handle of the implanter from the rear pushrod locking knob of the implanter to allow the front handle to be slidable relative to the rear pushrod, and pulling the front handle of the implanter backward towards the proximal end under the supervision of the second endoscope to allow the flexible tube to be moved backward together, so that the distal flange occluding body of the occluding stent is exposed and released from the flexible tube and is unfolded at the second side of the fistula in the second body channel; or pushing the rear handle forward towards the distal end to cause the front pusher to push the distal flange occluding body of the occluding stent out of the flexible tube, so that the distal flange occluding body of the occluding stent is released and unfolded at the second side of the fistula in the second body channel;
moving the over tube backward towards the proximal end to the flexible tube of the implanter to fully expose the distal end of the implanter, slowly moving the implanter backward towards the proximal end until the distal end of the flexible tube of the implanter is observed under the first endoscope to have returned to the first side of the fistula in the first body channel, pulling the front handle of the implanter backward towards the proximal end to cause the flexible tube to be moved backward together, so that the proximal flange occluding body of the occluding stent is exposed and released from the flexible tube and is unfolded at the first side of the fistula in the first body channel under the supervision of the first endoscope; or locking the front handle to the rear pushrod locking knob to cause the rear pushrod to be fixed to the front handle, and moving the implanter backward towards the proximal end, so that the distal flange occluding body is blocked by an inner wall of the second human second channel where the second side of the fistula is located and thus the proximal flange occluding body of the occluding stent is pulled out of the flexible tube, released and unfolded at the first side of the fistula in the first body channel; and
withdrawing the implanter to the outside of the body.

19. The method according to claim 18, wherein the first endoscope is an ultrafine gastroscope and the second endoscope is a tracheoscope.

20. The method according to claim 13, wherein:
locating the position of the fistula under an endoscope, wherein the fistula is located between the first body channel and the second body channel;
feeding the implanter through a clamp channel of an endoscope to allow the distal end of the implanter to pass through the fistula from the first side of the fistula in the first body channel to the second side of the fistula in the second body channel under the supervision of the endoscope, wherein the occluding stent is loaded in the flexible tube at the distal end of the implanter;
unlocking the front handle of the implanter from the rear actuator locking knob of the implanter to allow the front handle to be slidable relative to the rear pushrod, and pulling the front handle of the implanter backward towards the proximal end under the supervision of the endoscope to allow the flexible tube to be moved backward together, so that the distal flange occluding body of the occluding stent is exposed and released from the flexible tube and is unfolded at the second side of the fistula in the second body channel; or pushing the rear handle forward towards the distal end to cause the front pusher to push the distal flange occluding body of the occluding stent out of the flexible tube, so that the distal flange occluding body of the occluding stent is released and unfolded at the second side of the fistula in the second body channel;
slowly moving the implanter backward towards the proximal end until the distal end of the flexible tube of the implanter is observed under the endoscope to have returned to the first side of the fistula in the first body channel, pulling the front handle of the implanter backward towards the proximal end to cause the flexible tube to be moved backward together, so that the proximal flange occluding body of the occluding stent is exposed and released from the flexible tube and is unfolded at the first side of the fistula in the first body channel; or locking the front handle to the rear pushrod locking knob lock to cause the rear pushrod to be fixed to the front handle, and moving the implanter backward towards the proximal end, so that the distal flange occluding body is blocked by an inner wall of the second human second channel where the second side of the fistula is located and thus the proximal flange occluding body of the occluding stent is pulled out of the flexible tube, released and unfolded at the first side of the fistula in the first body channel; and
withdrawing the implanter to the outside of the body.
